(19) United States
(12) Patent Application Publication
(10) Pub. No.: US 2024/0336889 A1
(43) Pub. Date: Oct. 10, 2024

(54) NOMADIC BACTERIA AND USES THEREOF

(71) Applicants: The State of Israel, Ministry of Agriculture & Rural Development, Agricultural Research Organization, Rishon-LeZion (IL); Yissum Research Development Company of the Hebrew University of Jerusalem Ltd., Jerusalem (IL)

(72) Inventors: Moshe SHEMESH, ModiIn (IL); Satish Kumar RAJASEKHARAN, Rishon LeTsion (IL); Doron STEINBERG, Jerusalem (IL)

(73) Assignees: The State of Israel, Ministry of Agriculture & Rural Develoment, Agricultural Research Organization, Rishon-LeZion (IL); Yissum Research Development Company of the Hebrew University of Jerusalem Ltd., Jerusalem (IL)

(21) Appl. No.: 18/746,141

(22) Filed: Jun. 18, 2024

Related U.S. Application Data
(63) Continuation of application No. PCT/IL2022/051369, filed on Dec. 21, 2022.
(60) Provisional application No. 63/292,545, filed on Dec. 22, 2021.

(51) Int. Cl. C12N 1/20; A01N 63/22; C12R 1/125; C12R 1/245; C12R 1/25

(52) U.S. Cl. CPC C12N 1/205; A01N 63/22; C12R 2001/125; C12R 2001/245; C12R 2001/25

(57) ABSTRACT

A method of generating a biofilm of nomadic bacteria. The method comprises:
(a) culturing the nomadic bacteria in an acidic environment under conditions that promote generation of a V-type structure of the nomadic bacteria; and subsequently
(b) culturing said nomadic bacteria having a V-type structure on an adherent surface, thereby generating the biofilm comprising the nomadic bacteria. Conditioned media of nomadic bacteria are also disclosed and uses thereof.

### RELATED APPLICATIONS

This application is a Continuation of PCT Patent Application No. PCT/IL2022/051369 having International filing date of Dec. 21, 2022 which claims the benefit of priority under 35 USC § 119 (e) of U.S. Provisional Patent Application No. 63/292,545, filed on Dec. 22, 2021. The contents of the above applications are all incorporated by reference as if fully set forth herein in their entirety.

### FIELD AND BACKGROUND OF THE INVENTION

The present invention, in some embodiments thereof, relates to methods of culturing nomadic bacteria and conditioned medium generated therefrom in order to control biofilm formation.

Living microbial cells which are administered in adequate amounts, confer a beneficial physiological effect on the host, are known as “probiotics”. Studies have shown therapeutic effects that probiotic bacteria can provide to the host in maintaining a healthy gut and controlling several types of gastrointestinal infections. Due to their perceived health benefits, probiotic bacteria have been increasingly incorporated into a variety of food and drink products during the last few decades. Some of the most common types of microorganisms used as probiotics are the lactic acid bacteria (LAB), which mainly belong to the genera Lactobacillus and Bifidobacterium. Both these genera are dominant inhabitants in the human intestine and have a long history of safe use and are considered as GRAS (generally recognized as safe). To assure their beneficial effects in the body, these organisms must survive during food processing, storage and the passage through the upper gastrointestinal tract (GIT) and arrive alive to their site of action. However, previous studies have shown low survival level of probiotic bacteria in the final food product and a considerable loss in their viability to high acidic conditions of the stomach and high bile concentration in the small intestine. In addition, probiotics are usually available as dry bacterial powders prepared mainly by freeze drying which has been established as a procedure that may cause fatal injury to cells. Therefore, there is a need to develop novel technologies aimed to improve the survival of health-promoting bacteria during food production, as well as through the storage and ingestion processes in order to maintain delivery of probiotics to humans.

Lactobacillus plantarum is a prospective probiotic bacterium in the food and supplements industry. L. plantarum displays diverse morphological phenotypes and a remarkable ability to acclimatize to external settings, making it suitable for wide-ranging medicinal and industrial applications.

L. plantarum manifest a strong ability to auto-aggregate depending on external environments and nutrient availability. Auto-aggregation or co-aggregation is an exciting adaptation tactic that potentiates the probiotics to combat harmful pathogens. A probiotic Lactobacillus gasseri was shown to adhere to the human intestinal mucosa by autoaggregation, creating a protective blanket, which prevents pathogen colonization. In addition, several strains of L. plantarum are routinely assessed for their auto-aggregation abilities citing its effectiveness for pathogen control.

Tolerance to acidic conditions is yet another adaptation response manifested by Lactobacilli to curtail pathogen growth in a microbiome. In general, cells of L. plantarum are well-adapted to withstand diverse pH-induced stresses. They grow best at sub-optimal (pH 5.0) pH conditions, which triggers a pre-adaptation response eliciting the cells to swiftly adjust and respond against the same stress in a better way¹⁵. Other Lactobacilli are reported to tolerate extremely low-pH stresses by arresting growth and favouring colonization. For instance, L. acidophilus cultured in pH 3 revealed enhanced ability to survive and adhere to human intestinal cells¹⁶. Other studies have shown that the low pH (3.5-4.5) triggers L. plantarum upsurge in the human vaginal microbiome¹⁷. The proliferation, in turn, aids L. plantarum to exhibit complete dominance during competition with other vaginal pathogens. Overall, phenotypes displayed by L. plantarum in response to acidic-pH sheds light on the remarkable adaptability to survive and combat pathogens thereof, which seems sturdily connected to its origins and distribution.

### SUMMARY OF THE INVENTION

According to an aspect of the present invention there is provided a method of generating a biofilm comprising a nomadic bacteria, the method comprising:
(a) culturing the nomadic bacteria in an acidic environment under conditions that promote generation of a V-type structure of the nomadic bacteria; and subsequently
(b) culturing said nomadic bacteria having a V-type structure on an adherent surface, thereby generating the biofilm comprising the nomadic bacteria.

According to another aspect of the invention, there is provided a method of culturing nomadic bacteria comprising culturing the nomadic bacteria in an acidic environment under conditions that promote generation of a V-type structure of the nomadic bacteria for at least 24 hours, thereby culturing the nomadic bacteria.

According to an embodiment of the present invention, the. nomadic bacteria are of the Lactobacillus genus.

According to an embodiment of the present invention, the nomadic bacteria are of a species selected from the group consisting of L. rhamnosus, L. plantarum and L. casei.

According to an embodiment of the present invention, the nomadic bacteria is of the L. plantarum species and the acidic environment is between pH 2.5-pH 4.

According to an embodiment of the present invention, the nomadic bacteria is of the L. casei species and the acidic environment is between pH 3.5-pH 6.5.

According to an embodiment of the present invention, the adherent surface comprises a plastic or a glass.

According to an embodiment of the present invention, the adherent surface comprises a surface of a bioreactor.

According to an embodiment of the present invention, the method further comprises culturing an additional bacteria on the adherent surface so as to generate a biofilm comprising the nomadic bacteria and the additional bacteria.

According to an embodiment of the present invention, the additional bacteria comprises bacteria of the Bacillus species.

According to an embodiment of the present invention, the bacteria of the Bacillus species comprise Bacillus subtilis.

According to an aspect of the present invention there is provided a biofilm generated according to the method described herein.

According to an aspect of the present invention there is provided a composition comprising isolated, nomadic bacteria, wherein at least 30% of the bacteria have a V-shaped structure.

According to an embodiment of the present invention, the isolated, nomadic bacteria are nomadic bacteria.

According to an embodiment of the present invention, the isolated, nomadic bacteria are of the Lactobacillus genus.

According to an embodiment of the present invention, the isolated, nomadic bacteria are of a species selected from the group consisting of L. rhamnosus, L. plantarum and L. casei.

According to an embodiment of the present invention, the isolated, nomadic bacteria are freeze-dried.

According to an embodiment of the present invention, the composition is formulated as a solution, a suspension, an emulsion, a tablet, a granule, a powder, a capsule, a lozenge, a chewing gum, or a suppository.

According to an embodiment of the present invention, the composition is formulated in a food.

According to an aspect of the present invention there is provided a method of reducing biofilm formation of a pathogen comprising:
(a) culturing a nomadic bacteria in a medium subjected to at least one stress selected from the group consisting of a pH stress, a temperature stress, an oxidative stress, an osmotic stress and a combination thereof to generate a conditioned medium; and
(b) contacting the pathogen with the conditioned medium under conditions that reduce biofilm formation of the pathogen, thereby reducing biofilm formation of the pathogen.

According to an embodiment of the present invention, the pH stress is an acidic stress.

According to an embodiment of the present invention, the temperature stress is a cold stress.

According to an embodiment of the present invention, the combination comprises an acidic stress and a cold stress.

According to an embodiment of the present invention, the culturing promotes a chain structure of the nomadic bacteria.

According to an embodiment of the present invention, the culturing promotes a V-shape chain structure of the nomadic bacteria.

According to an embodiment of the present invention, the pathogen is a pathogenic bacteria.

According to an embodiment of the present invention, the pathogenic bacteria is E. coli or S. Aureus.

According to an embodiment of the present invention, the pathogen is a fungus.

According to an embodiment of the present invention, the fungus is Candida albicans.

According to an embodiment of the present invention, the contacting is effected in vivo.

According to an embodiment of the present invention, the contacting is effected ex vivo.

According to an embodiment of the present invention, the nomadic bacteria are of the lactobacillus genus.

According to an embodiment of the present invention, the nomadic bacteria are of a species selected from the group consisting of L. rhamnosus, L. plantarum and L. casei.

According to an aspect of the present invention there is provided a conditioned medium generated by culturing nomadic bacteria in a medium subjected to at least one stress selected from the group consisting of a pH stress, a temperature stress, an oxidative stress, an osmotic stress and a combination thereof.

According to an embodiment of the present invention, the culturing is effected on a non-adherent surface.

According to an embodiment of the present invention, the nomadic bacteria are of the lactobacillus genus.

According to an embodiment of the present invention, the nomadic bacteria are of a species selected from the group consisting of L. rhamnosus, L. plantarum and L. casei.

According to an embodiment of the present invention, the stress is a pH stress.

According to an embodiment of the present invention, the pH stress is an acidic stress.

According to an embodiment of the present invention, the at least one stress comprises an acid stress and a cold stress.

According to an embodiment of the present invention, the conditioned medium comprises 2-undecanone.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-F. Spatio-temporal establishment of conic-shaped colonies in L. plantarum.
A. Colonies of L. plantarum 3297 were generated onto the MRS hard agar plates (pH 5.5) following 2 and 7d incubation at 37° C. Scale bar: 0.2 cm.
B. Estimation of the L. plantarum 3297 colony heights generated on the MRS hard agar plates with different pH conditions. The graph shows the means±SEMs of three measurements. * P<0.05 vs. the control. Here, the colonies grown at pH 5.5 are taken as control compared to those grown at pH 7.
C. Effect of ΔpH on conic tip formation and colouration of the L. plantarum colonies. White and black arrows show the abolished tip. Scale bar: 0.1 cm.
D. The CFU quantitation of L. plantarum 3297 cells grown at pH 5.5 and 7 after 7 d of incubation on MRS hard agar. The graph shows the means±SEMs of three measurements. * P>0.05 vs. the control. Here, the colonies grown at pH 5.5 are taken as control compared to those grown at pH 7.
E. The CFU quantitation of L. plantarum 3297 cells exposed to desiccation stress. The cells were prior grown on MRS hard agar at pH 5.5 or 7 for 7d. After incubation, the conic colonies were directly transferred to the desiccation unit (at 40% relative humidity and 30° C. for 20 h). The graph shows the means±SEMs of three measurements. *** P<0.001 vs. the non-treated controls.
F. The CFU quantitation of L. plantarum 3297 cells exposed to desiccation stress. The cells were prior grown on MRS hard agar at pH 5.5 or 7 for 7d. After incubation, the conic colonies were directly transferred to the desiccation unit (at 40% relative humidity and 30° C. for 4 d). The graph shows the means±SEMs of three measurements. *** P<0.001 vs. the non-treated controls.

FIGS. 2A-E. Influence of cold stress on survivability of L. plantarum grown in conic colonies.
A. The CFU quantitation of L. plantarum 3297 conic colonies following exposure to cold stress. The cells were grown on MRS hard agar (pH 5.5) for 7d and transferred to cold conditions (−17° C.) for 1h. After incubation, the colonies were resuspended in phosphate-buffered saline (PBS), vortexed, diluted, and plated on fresh MRS agar plates. Similarly, the colonies were also incubated for 1 week at 4° C. and assessed. The graph shows the means±SEMs of three measurements. ** P<0.001 vs. the non-treated controls.
B. Dual staining (SYTO™ and propidium iodide (PI)) of consolidated bundles that was induced following frozen stress (−17° C., 1 h). Scale bar: 20 μm.
C. The CFU quantitation of L. plantarum 3297 conic colonies following exposure to frozen stress. The cells were grown on MRS hard agar pH 5.5 or 7 for 7d and transferred to cold conditions (−17° C.) for 1 h. After incubation, the colonies were resuspended in phosphate-buffered saline (PBS), vortexed, diluted, and plated on fresh MRS agar plates. The graph shows the means±SEMs of three measurements. *** P<0.001 vs. the non-treated controls.
D. Time-course analysis of cellular aggregation of L. plantarum 3297 cells from conic colonies to form consolidated bundles (in 1 h). Scale bar: 20 μm.
E. Schematic experimental setup that led to the sighting of consolidated bundles.

FIGS. 3A-D. The heat-shock proteins are involved in cold shock stress response
A. Consolidated bundles of wild-type L. plantarum WCFS1 or hsp1 mutant. Scale bar: 20 μm.
B. Colony forming units (CFUs) of wild-type L. plantarum WCFS1 or hsp1 mutant following exposure to frozen stress. The cells were grown on MRS hard agar pH 5.5 for 7d and transferred to cold conditions (−17° C.) for 1 h. After incubation, the colonies were resuspended in phosphate-buffered saline (PBS), vortexed, diluted, and plated on fresh MRS agar plates. The graph shows the means±SEMs of three measurements. *** P<0.001 vs. the non-treated controls.
C. Crystal violet quantification of biofilms formed by L. plantarum WCFS1, and HSP mutants (hsp1, hsp2 or hsp3) on polystyrene surface. The cells were incubated at 37° C. for 24 h without shaking, following which the cells were stained with 0.4% crystal violet and read at OD₅₉₅. The graph shows the means±SEMs of three measurements. *** P<0.001 vs. the non-treated controls. Here cells grown at pH 5.5 are taken as a control.
D. Mechanistic flowchart depicting the involvement of heat-shock protein response in governing the multifaceted traits manifested by L. plantarum in response to acidic-pH.

FIGS. 4A-F. Effect of CSCF from L. plantarum conic colonies on probiotic B. subtilis.
A. Effect of cold-shock colony filtrates (CSCF) on unstressed L. plantarum 3297 cells from conic colonies. Lithium chloride (5M) is chemical that prevent cellular aggregation. Scale bar: 20 μm.
B. Effect of CSCF (10% v/v) on the growth of B. subtilis WT 3610 incubated at 37° C. for 24 h at 150 rpm. The graph shows the means±SEMs of three measurements. * P>0.05 vs. the control.
C. Effect of CSCF (10% v/v) on pellicle formation of B. subtilis WT 3610 incubated at 30° C. for 48 h without shaking.
D. Assays of β-galactosidase activities by B. subtilis YC121 cells harboring P_{tapA}-lacZ transcriptional reporter. The graph shows the means±SEMs of three measurements. ** P<0.01, *** P<0.001 vs. the non-treated controls.
E. Effect of CSCF (10% v/v) on bundle formation in B. subtilis YC189, harboring a transcriptional reporter for matrix expression. White arrowhead indicates the biofilm bundles induced in treated conditions. Scale bar: 20 μm.
F. Effect of acid-shock colony filtrates ASCF (10% v/v) on bundle formation in B. subtilis YC189, harboring a transcriptional reporter for matrix expression. White arrowhead indicates the biofilm bundles induced in treated conditions. Scale bar: 20 μm.

FIGS. 5A-H. Effect of low pH on L. plantarum geometric structure, biofilm formation, and in vitro and in vivo antagonism against C. albicans.
A. Phenotypic appearance of L. plantarum 3297 cells grown at pH 3.5 or 5.5 in liquid MRS. White arrows indicate septation. Scale bar: 20 μm.
B. Growth curves of L. plantarum grown at either pH 3.5 or 5.5. The cells were incubated at 37° C. for 24 h.
C. Crystal violet quantification of L. plantarum biofilms on polystyrene surface. The cells were incubated at 37° C. for 24 h without shaking, following which the cells were stained with 0.4% crystal violet and read at OD₅₉₅. The graph shows the means±SEMs of three measurements. *** P<0.001 vs. the non-treated controls. Here cells grown at pH 5.5 are taken as a control.
D. Fluorescent microscopic images of L. plantarum biofilms on polystyrene surface stained with SYTO™ 9. Scale bar: 200 μm.
E. Effect of Live probiotic (L. plantarum cells grown at pH 3.5 or 5.5) on mature C. albicans biofilms in in vitro conditions. C. albicans biofilms were grown on polystyrene plates. After 8 h of incubation, the broth containing the planktonic cells were removed, replaced with PBS (controls) or live L. plantarum in PBS, and incubated again for 24 h. Biofilm formation was then assessed microscopically after staining with SYTO™ 9. Scale bar: 200 μm.
F. Survival rates of nematodes (that was prior fed with L. plantarum cells grown at pH 3.5 or 5.5 or E. coli OP50) infected with C. albicans. The graph shows the means±SEMs of three measurements. ** P<0.01, and *** P<0.001 vs. the E. coli OP50 controls, *** P<0.05 vs. L. plantarum at pH 5.5.
G. The microscopic image of adult C. elegans exposed to C. albicans and subsequently recused with E. coli OP50 (control) or L. plantarum (pH 3.5 or 5.5) feed. Inset shows C. albicans hyphae that ruptured and killed the nematode in E. coli OP50 fed group. Scale bar: 200 μm.
H. A scheme depicting the formation of V-shaped phenotypes and their pertinence.

FIGS. 6A-B. Assessment of L. plantarum colony parameters.
A. Estimation of L. plantarum colony diameters grown on MRS hard agar with different pH conditions. The graph shows the means±SEMs of three measurements. * P>0.05 vs. the non-treated controls.
B. Representative image showing height difference between colonies grown on pH 7 and pH 5.5 MRS hard agars. Scale bar: 0.2 cm.

FIGS. 7A-C. Effect of colony aging on brown coloration within the colony.
A. Microscopic image of aging colonies of L. plantarum 3297 that show increased accumulation of brown matrix during prolonged incubation. Scale bar: 0.2 cm.
B. Colony forming units (CFUs) of L. plantarum grown at pH 5.5 after 5, 7 and 15 days of incubation. Whole colony (with similar diameter) was lifted, diluted in PBS and plated again on MRS agar. ** P<0.01 vs. 5d colonies.
C. Quantitative profile of dead cells (stained with propidium iodide (PI)) in the aged conic colony of L. plantarum. The graph shows the means±SEMs of three measurements. *** P<0.001 vs. 5d colonies.

FIG. 8. Effect of colony aging on brown coloration within the colony. Microscopic images of reactive oxygen species (ROS) positive (green), PI positive (red) and live cells (unstained in merge) in an ageing colony. Scale bar: 20 μm.

FIG. 9. Mean diameters of the circular bundles formed by WT and hsp1 mutant. The graph shows the means±SEMs of three measurements. *** P<0.001 vs. WT control.

FIG. 10. Effect of heat treated (60° C. for 30 min) colony filtrates (CF) on circular bundle formation. Scale bar: 20 μm. CSCF stands for ‘cold-shock colony filtrate’.

FIG. 11. Growth curve analysis of B. subtilis in the presence and absence of CSCF. v/v denotes volume per volume.

FIGS. 12A-C. Effect of low pH on biofilm formation by L. plantarum.
A. Qualitative (i) and quantitative data (ii) showing the ratio of V-shaped and linear L. plantarum cells grown in liquid broth (pH 3.5, for 24 h at) 37° and transferred to slides for microscopy. Scale bar: 20 μm. Magnification: 1000× (100× objective×10× ocular). The graph shows the means±SEMs of three measurements. *** P<0.001 vs. the linear cells.
B. Qualitative (i) and quantitative data (ii) showing the ratio of V-shaped and linear L. plantarum cells grown directly as biofilms on glass coverslips. For this experiment, a glass coverslip was dropped on the surface of a 12-well microtiter plate and grown with the MRS media (pH 3.5). After incubation for 48 h, the slides were carefully taken out, washed, stained with SYTO9 and imaged with florescent microscope. Scale bar: 20 μm. Magnification: 1000× (100× objective×10× ocular). The graph shows the means±SEMs of three measurements. *** P<0.001 vs. the linear cells.
C. Microscopic image of cells grown directly on 24-well microtiter plates in liquid broth (pH 3.5, incubated for 48 h at) 37°. The biofilms on the polystyrene surface (before fluorescent staining, but after washing procedure) of the microtiter plate is shown. Black inset box and dashed lines show the biofilms at the centre following staining. The area is dense and it is not possible to distinguish the V-shapes. The red box and dashed lines show the biofilms on the periphery where there were less cells and here the V-shapes can be clearly distinguished. Scale bar: 100 μm. Magnification: 100× (10× objective×10× ocular).

FIG. 13. Survival rates of uninfected nematodes fed with E. coli OP50, and L. plantarum cells grown at pH 3.5 or 5.5. The graph shows the means±SEMs of three measurements. * P>0.05 vs. the control.

FIG. 14. Effect on live probiotics on survival of C. elegans infected with S. aureus. The graph shows the means±SEMs of three measurements. * P<0.05 significance for L. plantarum (pH 3.5) vs L. plantarum (pH 5.5), and ** P<0.01, *** P<0.01 vs. the E. coli control.

FIG. 15. A model depicting microbial stress recovery in V-shaped and regular cells.

FIGS. 16A-C. Effect of Bacillary postbiotics and live L. plantarum on physiology of bacterial pathogens.
A. Effect of the unstressed (UP) and the cold-stressed postbiotics (CSP) on E. coli biofilm formation. Biofilms were generated on polystyrene surfaces in the presence or absence of the postbiotics and visualised and quantified by crystal violet staining method. The graph shows the means±SEMs of three measurements. *** P<0.001 vs. the non-treated controls.
B. Effect of the UP and CSP on S. aureus biofilm formation. Biofilms were generated on polystyrene surfaces in the presence or absence of the postbiotics and visualised and quantified by crystal violet staining method. The graph shows the means±SEMs of three measurements. *** P<0.001 vs. the non-treated controls.
C. Fluorescent microscopic images of bacterial biofilms on polystyrene surfaces treated with UP or CSP (5% or 10% v/v). Scale bar: 200 μm.

FIGS. 17A-B. Effect of the postbiotics on quorum sensing controlled phenotype (swarming motility) of Escherichia coli
A. Effect of the UP or CSP (5% & 10% v/v) on dendrite swarming motility of E. coli.
B. The diameter of E. coli. swarm area in UP or CSP treated and non-treated controls. The graph shows the means±SEMs of three measurements. *** P<0.001 vs. the non-treated controls.

FIGS. 18A-D. Effect of the UP and CSP on growth, biofilms and pre-formed biofilms of Candida albicans.
A. Growth profile of C. albicans in the presence and absence of the UP or CSP (10% v/v).
B. Crystal violet quantifications of C. albicans biofilm formation on polystyrene surface in the presence and absence of UP or CSP (10% v/v). The graph shows the means±SEMs of three measurements. *** P<0.001 vs. the non-treated controls.
C. Effect of the UP or CSP (20% v/v) on pre-formed mature biofilms of C. albicans. The graph shows the means±SEMs of three measurements. * P<0.05 vs. the non-treated controls. The graph shows the means±SEMs of three measurements. *** P<0.05 vs. the non-treated controls.
D. Fluorescent microscopic images of C. albicans biofilms on polystyrene surfaces treated with UP or CSP (10% v/v). The microtiter plates containing 24 h C. albicans biofilms were stained with SYTO™ 9. Scale bar: 100 μm.

FIGS. 19A-B. Microscopic images of Candida albicans colonies and hyphal filaments
A. Effect of the UP or CSP (10% v/v) on hyphal protrusion from colony edges of C. albicans grown on potato dextrose agar (PDA). The plates containing colonies were incubated for 7 days at 37° C. Scale bar: 200 μm.
B. Effect of the UP or CSP (10% v/v) on yeast-to-hyphal transition in colony edges of C. albicans grown in RPMI-1640 media. Scale bar: 20 μm.

FIGS. 20A-C. Effect of 2-undecanone on C. albicans.
A. Growth profile of C. albicans in the presence and absence of 2-undecanone in RPMI medium incubated at 37° C. with 150 rpm for 24 h. The graph shows the means±SEMs of three measurements. ** P<0.01 vs. the non-treated controls.
B. Crystal violet quantifications of C. albicans biofilm formation on polystyrene surface in the presence and absence of 2-undecanone in PDB. The graph shows the means±SEMs of three measurements. *** P<0.001 and ** P<0.01 vs. the non-treated controls.
C. Effect of 2-undecanone on C. albicans hyphal filaments in RPMI media hyphal protrusion from colony edges of C. albicans, and. Scale bar: 200 μm.

FIGS. 21A-C. Assessment of toxicity profile of UP, CSP and 2-undecanone in a C. elegans model.
A. Effect of UP or CSP (10% v/v) on C. elegans survival. The graph shows the means±SEMs of three measurements. * P>0.05 vs. the non-treated controls.
B. Effect of 2-undecanone (10% v/v) on C. elegans survival. The graph shows the means±SEMs of three measurements. * P>0.05 vs. the non-treated controls.
C. Effects of 2-undecanone (0.01%) on survival of C. elegans exposed to C. albicans for a period of seven days. ** p<0.01 vs. the non-treated controls.

FIGS. 22A-B. Molecular interaction of ketones with hyphal wall protein 1 (Hwp1 protein).
A. The 3D interaction diagram showing interactions between ligands (2-undecanone, 2-nonanone, and 2-heptanone) with the active sites of hyphal wall protein 1 (Hwp1).
B. The 2D image showing interactions between ligands (2-undecanone, 2-nonanone, and 2-heptanone) with the active sites of hyphal wall protein 1 (Hwp1). Pink arrow represents the H-bonds.

FIGS. 23A-B. Morphological studies on different strains of L. plantarum at pH 6.5 and pH 3.5 (A) Rod-shape morphology of different L. plantarum strains at pH 6.5 (left panel); V-shaped morphology of L. plantarum at pH 3.5 (right panel); (B) High-resolution SEM images of L. plantarum 12422 at pH 6.5 (a) and pH 3.5 (b) (at 20,000× magnification) and (c) (at 40,000× magnification).

FIGS. 24A-C. Morphological, cell growth and cell viability analysis of L. plantarum 12422 (A) Confocal imaging of L. plantarum 12422 at pH 6.5 (upper panel) and pH 3.5 (lower panel) at different time-points; (B) Growth curve analysis of L. plantarum 12422 at pH 6.5 and pH 3.5; (C) Colony forming units per ml of L. plantarum 12422 at pH 6.5 AND pH 3.5.

FIGS. 25A-B. General metabolic analysis of L. plantarum 12422 (A) Evaluation of metabolic activity of L. plantarum 12422 at pH6.5 and pH 3.5 using XTT assay (B) Quantification of ATP levels of L. plantarum 12422 at pH6.5 and pH 3.5 using BacTitre Glo™ ATP assay.

FIGS. 26A-C. Cell Cycle analysis of L. plantarum 12422. (A) Cell count of DAPI stained cells at 5 hours and 24 hours; pH 6.5 cells (left panel); pH3.5 (right panel) (B) Scatter plot of pH 6.5 and pH 3.5 DAPI stained cells (C) Confocal imaging of DAPI stained cells (a) pH 6.5 (b) pH 3.5 (c) Phase contrast image focused on V-shape cell at pH 3.5 (d) DAPI staining focused on V-shape cell.

FIGS. 27A-C. Gene expression analysis using Quantitative Real Time PCR. Selected set of genes involved in (A) Metabolism (B) Cell division and autolysins (C) Quorum sensing.

### DESCRIPTION OF SPECIFIC EMBODIMENTS OF THE INVENTION

The present invention, in some embodiments thereof, relates to methods of culturing nomadic bacteria and conditioned medium generated therefrom in order to control biofilm formation.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The probiotic bacterium Lactobacillus plantarum is often considered a ‘generalist’ by virtue of its bewildering yet astounding ability to adapt and survive under diverse conditions. Whilst studying the effect of some of these conditions on the bacterium, the present inventors uncovered a unique geometrical arrangement of multicellular community of L. plantarum cells. Particularly, a phenomenon of cone-shaped colonies and V-shaped cell chains were discovered in response to acidic-pH stress. After 24 hours of growth under acidic pH conditions, the bacteria in the V-shaped structures showed an enhanced metabolic activity and increase in ATP concentration (FIGS. 25A-B). Whereas cell viability decreased at pH 6.5, after 10 hours of culture, cell viability reached a steady state which remained constant at pH 3.5. This indicates that culturing V-shape producing bacteria under acidic conditions may be preferable when a large amount of viable bacteria is required.

Moreover, subsequent cold-stress response revealed a cellular sacrifice-for-survival phenomenon (FIGS. 2A-C) triggering a consolidated bundles formation, which appeared to be independently governed by a small heat shock protein 1 (HSP 1) and at least partially by a luxS quorum sensing system. Whilst further reducing the present invention to practice, the present inventors showed that live L. plantarum, and its conic colony filtrates displayed remarkable antagonistic activities against several microbial pathogens and, specifically, Candida albicans, a pathogenic yeast, in both in vitro (FIG. 5E) and in vivo Caenorhabditis elegans models (FIGS. 5F-G).

Thus, according to a first aspect of the present invention, there is provided a method of generating a biofilm comprising nomadic bacteria, the method comprising:
(a) culturing the nomadic bacteria in an acidic environment under conditions that promote generation of a V-type structure of the nomadic bacteria; and subsequently
(b) culturing the nomadic bacteria having a V-type structure on an adherent surface, thereby generating the biofilm comprising the nomadic bacteria.

According to another aspect of the present invention, there is provided a method of culturing nomadic bacteria comprising culturing the nomadic bacteria in an acidic environment under conditions that promote generation of a V-type structure of the nomadic bacteria for at least 24 hours, thereby culturing the nomadic bacteria.

The term “nomadic bacteria” as used herein refers to bacteria which are capable of thriving in different ecological niches. In one embodiment, the nomadic bacteria are capable of surviving (i.e. propagating) in a pH from pH 2.5-pH 8. In another embodiment, the nomadic bacteria are capable of surviving (i.e. propagating) at a range of temperatures which are about 30° apart (e.g. from about 20° to about 50° C. In another embodiment, the nomadic bacteria are capable of surviving (i.e. propagating) in a dry environment and a humid environment.

According to a preferred embodiment, the nomadic bacteria of this aspect of the present invention belong to the genus lactobacillus. Exemplary species of nomadic lactobacillus contemplated by the present invention include but are not limited to L. rhamnosus, L. plantarum and L. casei.

In one particular embodiment, the species of lactobacillus is L. plantarum.

According to a particular embodiment, a single species of nomadic bacteria is cultured per culture under the acidic conditions described herein. According to another embodiment, no more than two, three, four or five species of nomadic bacteria are cultured in a single culture under the acidic conditions described herein.

Any number of strains of nomadic bacteria may be cultured in a single culture under acid conditions, as described herein. In one embodiment, no more than 500 different strains of nomadic bacteria are cultured in a single culture, no more than 250 different strains of nomadic bacteria are cultured in a single culture, no more than 100 different strains of nomadic bacteria are cultured in a single culture, no more than 90 different strains of nomadic bacteria are cultured in a single culture, no more than 80 different strains of nomadic bacteria are cultured in a single culture, no more than 70 different strains of nomadic bacteria are cultured in a single culture, no more than 60 different strains of nomadic bacteria are cultured in a single culture, no more than 50 different strains of nomadic bacteria are cultured in a single culture, no more than 40 different strains of nomadic bacteria are cultured in a single culture, no more than 30 different strains of nomadic bacteria are cultured in a single culture, no more than 20 different strains of nomadic bacteria are cultured in a single culture, no more than 10 different strains of nomadic bacteria are cultured in a single culture, no more than 9 different strains of nomadic bacteria are cultured in a single culture, no more than 8 different strains of nomadic bacteria are cultured in a single culture, no more than 7 different strains of nomadic bacteria are cultured in a single culture, no more than 6 different strains of nomadic bacteria are cultured in a single culture, no more than 5 different strains of nomadic bacteria are cultured in a single culture, no more than 4 different strains of nomadic bacteria are cultured in a single culture, no more than 3 different strains of nomadic bacteria are cultured in a single culture, no more than 2 different strains of nomadic bacteria are cultured in a single culture only one strain of nomadic bacteria is cultured per single culture.

In one embodiment, the nomadic bacteria, (following ingestion) promote the health of a human being. In another embodiment, the nomadic bacteria are used in industry to generate a product that is useful for human beings (e.g. methane, petroleum, insecticide etc.). In another embodiment, the nomadic bacteria are used in the food industry. In another embodiment, the nomadic bacteria are used in a silage inoculant. In still another embodiment, the nomadic bacteria are used in agriculture to support the growth of plants. In still another embodiment, the nomadic bacteria are used in bioremediation.

In one embodiment, the nomadic bacteria are probiotic bacteria.

The term “probiotic bacteria” as used herein refers to live bacteria which when administered in adequate amounts confer a health benefit on the host (e.g. human).

Among the principal mechanisms of probiotic action, it is possible to find the inhibition of enteric pathogens by the production of lactic acid, hydrogen peroxide and bacteriocins; competitive exclusion of enteric pathogens by blocking adhesion sites, competition for nutrients and modulation of the immune system, including inflammation reduction. They also provide benefits to the host, such as lactose intolerance alleviation; cholesterol decrease by assimilation, sustenance of the intestinal normal microbiota and dysbiosis ameliorating suppression of toxin production, degradation of toxin receptors in the intestine, preservation of normal intestinal pH, increase intestinal motility and help to maintain the integrity of the intestine permeability.

As mentioned, the nomadic bacteria are cultured under acidic conditions that promote generation of a V-type structure.

The phrase “V-type structure” as used herein refers to a particular type of cell chaining where several (typically four partially separated) cells form a filament with V-shaped curvature. This type of chaining can be identified using either light microscopy or SEM.

Typically, the nomadic bacteria are cultured for at least 20 hours so as to promote the V-type structure formation.

According to a particular embodiment, the nomadic bacteria are cultured for at least 24 hours, at least 48 hours, at least one week, at least two weeks or longer. For long term-culture (i.e. longer than 24 hours), culturing in a bioreactor may be preferable.

The culturing may be carried out on a liquid medium or a solid medium (e.g. further comprises a gelling agent).

Examples of gelling agents contemplated by the present invention include, but are not limited to agar, guar gum, xanthan gum, locust bean gum, gellan gum, polyvinyl alcohol, alkylcellulose, carboxyalkylcellulose and hydroxyalkylcellulose.

According to a specific embodiment, the culturing is not effected on an adherent surface.

In another embodiment, the culturing is effected on an adherent surface.

Exemplary media for culturing L. rhamnosus, L. plantarum and L. casei include MRS and M17 media, as well as skim milk.

The present inventors have shown that culturing of L. plantarum species in an acidic environment of pH 3.5 brings about formation of the V-type structure. Thus, the present inventors contemplate culturing L. plantarum in an acidic environment between pH 2.5-pH 4 to direct formation of the V-type structure.

In addition, culturing of L. casei species in an acidic environment between pH 3.5-pH 6.5 brings about formation of the V-type structure.

In one embodiment, the nomadic bacteria are cultured at a temperature between 30-40° C. (for example about 37° C.) during formation of the V-type structure.

Preferably, the culturing is carried out for a length of time such that at least 30%, 40%, 50%, 60%, 70%, 80%, 90% of the bacteria have a V-type structure. In one embodiment, the length of time is at least 12 hours and more preferably about 24 hours, 48 hours, at least one week, at least two weeks.

Once the V-type structures are formed, the bacteria may be isolated from the culture.

Thus, according to another aspect of the present invention there is provided a composition comprising isolated, nomadic bacteria, wherein at least 30% of the bacteria have a V-shaped structure.

The isolated V-shaped bacteria may be subject to drying (i.e. dehydrated), freezing, spray drying, or freeze-drying. Preferably, the V-shaped bacteria are treated in a way that preserves the viability of the bacteria.

In some embodiments, the V-shaped bacteria are formulated in the form of a suspension, an emulsion, a tablet, a granule, a powder, a capsule, a lozenge, a chewing gum, a suppository a powder or a liquid. If provided as a powder, combining the powder with a suitable liquid (e.g., liquid dairy product, fruit or vegetable juice, blended fruit or vegetable juice product, etc.) is specifically contemplated.

The isolated V-type structured nomadic bacteria have an enhanced ability to form biofilm. Thus, typically the amount of biofilm generated with the V-type structured nomadic bacteria is at least twice the amount of biofilm generated with the identical bacteria which does not have the V-type structure (for example prior to the acid exposure).

In one embodiment, the V type structures are removed from the acidic culture and subsequently cultured on an adherent surface (in a non-acidic environment). In another embodiment, the V-type structures are generated on an adherent surface and the generation of biofilm is initiated by increasing the pH.

Exemplary adherent surfaces on which the culturing can be carried out include a wide range of substrates, ranging from various polymeric materials (silicone, polystyrene, polyurethane, and epoxy resins) to metals and metal oxides (silicon, titanium, aluminum, silica, and gold) and glass. Fabrication techniques (soft lithography and double casting molding techniques, microcontact printing, electron beam lithography, nanoimprint lithography, photolithography, electrodeposition methods, etc.) can be carried out on such materials in order to alter the topography of the solid surface.

In one embodiment, the adherent surface is a surface of a bioreactor.

As used herein, the term “bioreactor” refers to an apparatus adapted to support biofilm of the invention.

The bioreactor will generally comprise one or more supports for the biofilm which may form a film thereover, and wherein the support is adapted to provide a significant surface area to enhance the formation of the biofilm. The bioreactors of the invention may be adapted for continuous throughput.

Culturing on the adherent surface is typically effected under conditions that allow the nomadic bacteria to retain their ability to form biofilms and preferably to retain their V-type structure. Typically the pH for culturing the V-type structures as biofilms is between pH 4-8.

It will be appreciated that additional bacteria may be co-cultured with the nomadic bacteria having a V-type structure to generate the biofilm.

The additional bacteria may be beneficial bacteria.

As used herein the term “beneficial bacteria” refers to any bacteria that bring about a positive effect on human beings.

The additional bacteria may be biofilm producing bacteria.

In one embodiment, the beneficial bacteria, when ingested promote the health of a human being. In another embodiment, the beneficial bacteria are used in industry to generate a product that is useful for human beings (e.g. methane, petroleum, insecticide etc.). In another embodiment, the beneficial bacteria are used in the food industry. In another embodiment, the beneficial bacteria are used in a silage inoculant. In still another embodiment, the beneficial bacteria are used in agriculture to support the growth of plants. In still another embodiment, the beneficial bacteria are used in bioremediation.

In one embodiment, the beneficial bacteria are probiotic bacteria.

In one embodiment, the beneficial bacteria are of the genus Bacillus, e.g. of the species Bacillus subtilis, Bacillus sonorensis, Bacillus licheniformis, Bacilllus firmus, Bacillus megaterium, B. endophyticus, Bacillus endophyticus and Bacillus amyloliquefaciens.

According to a particular embodiment, the species is Bacillus subtilis.

The biofilm generated according to the methods described herein (or the isolated V-type structure bacteria) may be used in a probiotic composition.

In some embodiments, the probiotic composition comprises from about 10³ to 10¹⁵ colony forming units (“CFUs”) of the nomadic bacteria per gram of finished product. In some embodiments, the probiotic composition comprises from about 10⁴ to about 10¹⁴ CFUs of the nomadic bacteria per gram of finished product. In some embodiments, the probiotic composition comprise from about 10⁵ to about 10¹⁵ CFUs of nomadic bacteria per gram of finished product. In some embodiments, the probiotic composition comprises from about 10⁶ to 10¹¹ colony forming units of the nomadic bacteria per gram of finished product. In some embodiments, the probiotic composition comprises from about 10² to about 10⁵ colony forming units of nomadic bacteria per gram of finished product.

It will be appreciated that at least 20%, 30%, 40%, 50%, 60%, 70%, 80%, 90% or more of the nomadic bacteria of the composition are viable (i.e. propagate).

The probiotic composition may comprise additional beneficial bacteria such as those belonging to the Bifidobacterium genus. Contemplated species of Bifidobacterium that may be present in the probiotic composition of this aspect of the present invention include, but are not limited to Bifidobacterium longum, Bifidobacterium bifidum, Bifidobacterium breve, Bifidobacterium infantis, Bifidobacterium adolecentis, Bifidobacterium lactis, and Bifidobacterium animalis. In some embodiments, the probiotic composition comprises a species that belongs to the genus lactobacillus e.g. Lactobacillus plantarum and at least two microorganisms selected from the following Bifidobacterium longum, Bifidobacterium bifidum, Bifidobacterium breve, Bifidobacterium infantis, Bifidobacterium adolecentis, Bifidobacterium lactis, and Bifidobacterium animalis.

In one embodiment, the bacterial compositions disclosed herein are in any form suitable for administering the composition to a mammalian subject. In some embodiments, the composition is in the form of a tablet, a powder or a liquid. If provided as a powder, combining the powder with a suitable liquid (e.g., liquid dairy product, fruit or vegetable juice, blended fruit or vegetable juice product, etc.) is specifically contemplated.

In some embodiments, the bacterial compositions disclosed herein are administered to a subject prior to, concomitant with or following administration of an antibiotic agent.

In some embodiment, the bacterial compositions described herein are formulated for topical administration—e.g. in a cream, a gel, a lotion, a shampoo, a rinse. The bacterial compositions may be administered to the skin or the scalp. The bacterial compositions may be useful for dental applications. For such applications they may be administered to the gums.

In some embodiments the compositions described herein are incorporated into a food product. The term “food product” as used herein refers to any substance containing nutrients that can be ingested by an organism to produce energy, promote health and wellness, stimulate growth, and maintain life. The term “enriched food product” as used herein refers to a food product that has been modified to include the composition comprising composition described herein, which provides a benefit such as a health/wellness-promoting and/or disease-preventing/mitigating/treating property beyond the basic function of supplying nutrients.

The probiotic composition can be incorporated into any food product. Exemplary food products include, but are not limited to, protein powder (meal shakes), baked goods (cakes, cookies, crackers, breads, scones and muffins), dairy-type products (including but not limited to cheese, yogurt, custards, rice pudding, mousses, ice cream, frozen yogurt, frozen custard), desserts (including, but not limited to, sherbet, sorbet, water-ices, granitas and frozen fruit purees), spreads/margarines, pasta products and other cereal products, meal replacement products, nutrition bars, trail mix, granola, beverages (including, but not limited to, smoothies, water or dairy beverages and soy-based beverages), and breakfast type cereal products such as oatmeal. For beverages, the probiotic composition described herein may be in solution, suspended, emulsified or present as a solid.

In one embodiment, the enriched food product is a meal replacement product. The term “meal replacement product” as used herein refers to an enriched food product that is intended to be eaten in place of a normal meal. Nutrition bars and beverages that are intended to constitute a meal replacement are types of meal replacement products. The term also includes products which are eaten as part of a meal replacement weight loss or weight control plan, for example snack products which are not intended to replace a whole meal by themselves, but which may be used with other such products to replace a meal or which are otherwise intended to be used in the plan. These latter products typically have a calorie content in the range of from 50-500 kilocalories per serving.

In another embodiment, the food product is a dietary supplement. The term “dietary supplement” as used herein refers to a substance taken by mouth that contains a “dietary ingredient” intended to supplement the diet. The term “dietary ingredients” includes, but is not limited to, the composition comprising the probiotic composition as described herein as well as vitamins, minerals, herbs or other botanicals, amino acids, and substances such as enzymes, organ tissues, glandulars, and metabolites.

In yet another embodiment, the food product is a medical food. The term “medical food” as used herein means a food which is formulated to be consumed or administered entirely under the supervision of a physician and which is intended for the specific dietary management of a disease or condition for which distinctive nutritional requirements, based on recognized scientific principles, are established by medical evaluation.

It is also well established that the addition of probiotic microorganisms to animal feed can improve animal efficiency and health. Specific examples include increased weight gain-to-feed intake ratio (feed efficiency), improved average daily weight gain, improved milk yield, and improved milk composition by dairy cows as described by U.S. Pat. Nos. 5,529,793 and 5,534,271. The administration of probiotic organisms can also reduce the incidence of pathogenic organisms in cattle, as reported by U.S. Pat. No. 7,063,836.

Thus, according to another embodiment, the probiotic composition described herein can be incorporated into an animal feed.

In one embodiment, the probiotic composition is designed for continual or periodic administration to ruminal, cecal or intestinal fermentors throughout the feeding period in order to reduce the incidence and severity of diarrhea and/or overall health. In this embodiment, the probiotic composition can be introduced into the rumen, cecum and/or intestines of the animal.

In yet another embodiment, the probiotic composition described herein are incorporated into a pharmaceutical product or composition. Pharmaceutical compositions comprise a prophylactically or therapeutically effective amount of the composition described herein and typically one or more pharmaceutically acceptable carriers or excipients (which are discussed below).

The disclosure contemplates formulations of the bacterial compositions described herein that are, in some embodiments, powdered, tableted, encapsulated or otherwise formulated for oral administration. The compositions may be provided as pharmaceutical compositions, nutraceutical compositions (e.g., a dietary supplement), or as a food or beverage additive, as defined by the U.S. Food and Drug Administration. The dosage form for the above compositions are not particularly restricted. For example, liquid solutions, suspensions, emulsions, tablets, pills, capsules, sustained release formulations, powders, suppositories, liposomes, microparticles, microcapsules, sterile isotonic aqueous buffer solutions, and the like are all contemplated as suitable dosage forms.

The compositions typically include one or more suitable diluents, fillers, salts, disintegrants, binders, lubricants, glidants, wetting agents, controlled release matrices, colorings, flavoring, carriers, excipients, buffers, stabilizers, solubilizers, commercial adjuvants, and/or other additives known in the art.

Any pharmaceutically acceptable (i.e., sterile and acceptably non-toxic as known in the art) liquid, semisolid, or solid diluent that serves as a pharmaceutical vehicle, excipient, or medium can be used. Exemplary diluents include, but are not limited to, polyoxyethylene sorbitan monolaurate, magnesium stearate, calcium phosphate, mineral oil, cocoa butter, and oil of theobroma, methyl- and propylhydroxybenzoate, talc, alginates, carbohydrates, especially mannitol, alpha.-lactose, anhydrous lactose, cellulose, sucrose, dextrose, sorbitol, modified dextrans, gum acacia, and starch.

Pharmaceutically acceptable fillers can include, for example, lactose, microcrystalline cellulose, dicalcium phosphate, tricalcium phosphate, calcium sulfate, dextrose, mannitol, and/or sucrose. Salts, including calcium triphosphate, magnesium carbonate, and sodium chloride, may also be used as fillers in the pharmaceutical compositions.

Binders may be used to hold the composition together to form a hard tablet. Exemplary binders include materials from organic products such as acacia, tragacanth, starch and gelatin. Other suitable binders include methyl cellulose (MC), ethyl cellulose (EC) and carboxymethyl cellulose (CMC).

In some embodiments, an enriched food product further comprises a bioavailability enhancer, which acts to increase the absorption of the sorbable natural product(s) by the body. Bioavailability enhancers can be natural or synthetic compounds. In one embodiment, the enriched food product comprising the composition described herein further comprises one or more bioavailability enhancers in order to enhance the bioavailability of the bioactive natural product(s).

Natural bioavailability enhancers include ginger, caraway extracts, pepper extracts and chitosan. The active compounds in ginger include 6-gingerol and 6-shogoal. Caraway oil can also be used as a bioavailability enhancer (U.S. Patent Application 2003/022838). Piperine is a compound derived from pepper (Piper nigrum or Piper longum) that acts as a bioavailability enhancer (see U.S. Pat. No. 5,744,161). Piperine is available commercially under the brand name Bioperine® (Sabinsa Corp., Piscataway, N.J.). In some embodiments, the natural bioavailability enhancers is present in an amount of from about 0.02% to about 0.6% by weight based on the total weight of enriched food product.

Examples of suitable synthetic bioavailability enhancers include, but are not limited to surfactants including those composed of PEG-esters such as are commercially available under the tradenames: Gelucire®, Labrafil®, Labrasol®, Lauroglycol®, Pleurol Oleique® (Gattefosse Corp., Paramus, N.J.) and Capmul® (Abitec Corp., Columbus, Ohio).

The amount and administration regimen of the composition is based on various factors relevant to the purpose of administration, for example human or animal age, sex, body weight, hormone levels, or other nutritional need of the human or animal. In some embodiments, the composition is administered to a mammalian subject in an amount from about 0.001 mg/kg body weight to about 1 g/kg body weight.

A typical regimen may comprise multiple doses of the composition. In one embodiment, the composition is administered once per day. The composition may be administered to an individual at any time. In some embodiments, the composition is administered concurrently, or prior to or at the consumption of a meal.

In some embodiments the bacterial compositions of this aspect of the present invention are formulated for use as an agricultural product. The bacterial compositions may be added to an agricultural carrier such as soil or plant growth medium. Other agricultural carriers that may be used include fertilizers, plant-based oils, humectants, or combinations thereof. Alternatively, the agricultural carrier may be a solid, such as diatomaceous earth, loam, silica, alginate, clay, bentonite, vermiculite, seed cases, other plant and animal products, or combinations, including granules, pellets, or suspensions. Mixtures of any of the aforementioned ingredients are also contemplated as carriers, such as but not limited to, pesta (flour and kaolin clay), agar or flour-based pellets in loam, sand, or clay, etc. Formulations may include food sources for the cultured organisms, such as barley, rice, or other biological materials such as seed, leaf, root, plant elements, sugar cane bagasse, hulls or stalks from grain processing, ground plant material or wood from building site refuse, sawdust or small fibers from recycling of paper, fabric, or wood. Other suitable formulations will be known to those skilled in the art.

In one embodiment, the agricultural formulation comprises a fertilizer. Preferably, the fertilizer is one that does not reduce the viability of the bacterial composition by more than 20%, 30%, 40%, 50% or more.

In some cases, it is advantageous for the agricultural formulation to contain agents such as herbicide, a nematicide, an insecticide, a plant growth regulator, a rodenticide, and a nutrient. Such agents are ideally compatible with the plant onto which the formulation is applied (e.g., it should not be deleterious to the growth or health of the plant). Furthermore, the agent is ideally one which does not cause safety concerns for human, animal or industrial use (e.g., no safety issues, or the compound is sufficiently labile that the commodity plant product derived from the plant contains negligible amounts of the compound).

The present inventors also contemplate that the presently disclosed agricultural composition may be comprised in an article of manufacture which further comprises an agent which promotes the growth of plants.

The agents may be formulated together with the nomadic bacteria in a single composition, or alternatively packaged separately, but in a single container.

Suitable agents are described herein above. Other suitable agents include fertilizers, pesticides (an herbicide, a nematocide, a fungicide and/or an insecticide), a plant growth regulator, a rodenticide, and a nutrient, as further described herein below.

In one embodiment, the agent which promotes the growth of the plant lacks anti-bacterial activity.

The present inventors have further shown that nomadic bacteria cultured under stress secrete compounds into the culture medium which aid in reducing biofilm formation. Accordingly, the present inventors conceive that conditioned medium of nomadic bacteria cultured under stress can be used to reduce biofilm formation of pathogens.

Thus, according to another aspect of the present invention there is provided a method of reducing biofilm formation of a pathogen comprising:
(a) culturing a nomadic bacteria in a medium subjected to at least one stress selected from the group consisting of a pH stress, a temperature stress, an oxidative stress, an osmotic stress and a combination thereof to generate a conditioned medium; and
(b) contacting the pathogen with the conditioned medium under conditions that reduce biofilm formation of the pathogen, thereby reducing biofilm formation of the pathogen.

According to this aspect nomadic bacteria (as described herein above) are cultured in a liquid medium under at least one stress to generate a conditioned medium.

Conditioned medium is the growth medium of a cell culture following a certain culturing period. The conditioned medium may include metabolites, organic acids, bacteriocins, antimicrobial peptides growth factors and cytokines secreted by the cells in the culture.

Such a growth medium can be any medium suitable for culturing the nomadic, bacterial cells. The growth medium can be supplemented with nutritional factors, such as amino acids, (e.g., L-glutamine), anti-oxidants (e.g., beta-mercaptocthanol) and growth factors, which benefit bacterial cell growth.

During culture, the nomadic bacterial cells are subjected to a stress, non-limiting examples of which include pH stress (e.g. acid stress), a temperature stress (e.g. cold stress), an oxidative stress and an osmotic stress. In one embodiment, the nomadic bacterial cells are subjected to more than one stress (e.g. initially a pH stress and subsequently a temperature stress). Thus, for example the present inventors contemplate subjecting nomadic cells (e.g. L. plantarum) cells initially to an acid stress between pH 3.5-6.5 and subsequently a cold stress of 0° C.-−20° C. for about 1 hour.

The nomadic bacterial cells are cultured in the growth medium for sufficient time to allow adequate accumulation of secreted factors that reduce biofilm formation of the pathogen. Furthermore, the conditions could be selected such that the nomadic bacterial cells form a 3D structure (e.g. a chain structure, of typically more than 4 bacterial cells forming an irregular shape or a V-type structure).

Typically, the medium is conditioned by culturing for 4-48 hours at 37° C. However, the culturing period can be scaled by assessing the effect of the conditioned medium on biofilm formation of the pathogen.

Selection of culture apparatus for conditioning the medium is based on the scale and purpose of the conditioned medium. Large-scale production preferably involves the use of dedicated devices. According to a particular embodiment, the conditioned medium is prepared in flasks. Continuous cell culture systems are reviewed in Furey (2000) Genetic Eng. News 20:10.

Typically, the culturing is effected on a non-adherent surface (e.g. not on glass or polystyrene plates).

Following accumulation of adequate factors in the medium, the conditioned medium is separated from the nomadic bacterial cells and collected. It will be appreciated that the nomadic feeder cells can be used repeatedly to condition further batches of medium over additional culture periods, provided that the cells retain their ability to condition the medium.

Presence of anti-biofilm agents in the conditioned medium may be verified prior to its use. In a particular embodiment, the presence of 2-undecanone may be established.

Once collected (and optionally, once presence of anti-biofilm activity has been verified), the conditioned medium may be contacted with a pathogen under conditions which reduce formation of a biofilm by the pathogen.

The term “pathogen” as used herein refers to a biofilm producing organism that causes disease. In one embodiment, the pathogen causes a disease in humans.

According to a specific embodiment the biofilm producing pathogen is a bacteria.

Particular examples of pathogenic biofilm-producing bacteria include E. coli, Listeria monocytogenes, Salmonella Enteritidis, Pseudomonas aeruginosa, Bacillus cereus, Streptococcus pyogenes, Staphylococcus epidermidis and S. aureus.

Table 1, herein below provides a summary of diseases which are caused by biofilm producing bacteria. The conditioned medium (or compounds isolated therefrom) may be used for treating such diseases.

TABLE 1

| Body System | Affected Organs | Disease |
|---|---|---|
| Auditory | Middle ear | Otitis media |
| Cardiovascular | Cardiac valves | Infective |
| | | endocarditis |
| | Arteries | Atherosclerosis |
| Digestive | Salivary glands | Sialolithiasis |
| | | (salivary duct |
| | | stones) |
| | Gall bladder | Recalcitrant |
| | | typhoid fever and |
| | | predisposition to |
| | | hepatobiliary |
| | | cancers |
| | Gastrointestinal | Inflammatory bowel |
| | tract, especially | disease |
| | the small and | and colorectal |
| | large intestine | cancer |
| Integumentary | Skin and | Wound |
| | underlying tissue | infections |
| Reproductive | Vagina | Bacterial |
| | | vaginosis |
| | Uterus and | Chronic |
| | fallopian tubes | endometritis |
| | Mammary glands | Mastitis |
| | (breasts) | |
| Respiratory | Nasal cavity and | Chronic |
| | paranasal sinuses | rhinosinusitis |
| | Throat, i.e., | Pharyngitis |
| | pharynx with | and |
| | tonsils and | laryngitis |
| | adenoids, and | |
| | larynx with | |
| | vocal cords | |
| | Upper and | Pertussis |
| | lower airways | (whooping cough) and |
| | | other Bordertella |
| | | infections |
| | Upper and lower | Cystic |
| | airways | fibrosis |
| Urinary | Prostate gland | Chronic bacterial |
| | | prostatitis |
| | Urethra, bladder, | Urinary tract |
| | urethers, kidneys | infections |

According to another embodiment, the biofilm producing pathogenic organism is a fungus (e.g. Candida albicans).

For treatment of diseases, the contacting may be in vivo or ex vivo.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Generally, the nomenclature used herein, and the laboratory procedures utilized in the present invention include molecular, biochemical, microbiological and recombinant DNA techniques. Such techniques are thoroughly explained in the literature.

### Example 1

### Spatiotemporal Establishment of Conic Colonies is Governed by an Adaptation Response of L. plantarum to Acidic-pH

### Materials and Methods

Microbial strains and culture media: Specifics of microbial strains used in the study are described in Table 1.

TABLE 1

| | | |
|---|---|---|
| B. subtilis NCIB3610 | Wild-type strain | (Branda, et al., 2001) |
| B. subtilis YC189 | P_{tapA-cfp} | (Chai, et al., 2011) |
| | in 3610 | |
| B. subtilis YC121 | P_{tapA-}LacZ | (Chen et al., 2015) |
| | in 3610 | |
| L. planarum 3297 | Isolate from | This study |
| | healthy cow | |
| C. albicans SC5314 | Clinical | (Feldman et al., 2017) |
| | specimen - human | |
| E. coli OP50 | Uracil auxotroph, | (Sanadaya et al., 2018) |
| | Feed for | |
| | C. elegans | |

Microbial strains were cultured and maintained in their respective selective media. For instance, L. plantarum was cultured, maintained and experimented in De Man, Rogosa and Sharpe (MRS) (HI media Pvt. Ltd. India) hard agar and/or liquid medium incubated at 37° C., non-shaking conditions, B. subtilis in LB (BD Difco, US) (37° C., 150 rpm for 5 h), and C. albicans in potato dextrose broth (PDB) (BD Difco, US), (37° C., 150 rpm, overnight) or PDB supplemented with agar, and/or Roswell Park Memorial Institute medium-1640 (RPMI) (Gibco, US) medium. The colonies were generated by setting the overnight culture to OD600=1 and achieving 10-7 dilutions that were subsequently spread on MRS hard agar plates and incubated for 5, 7, and 15 days at 37° C.

Growth curve analysis: Growth curve analysis was performed by procedures as previously described⁴⁰. L. Plantarum or B. subtilis cells were grown overnight in MRS or LB, respectively, using incubation conditions described above. The cultures were diluted 1:100 into new MRS (pH 3.5 or 5.5) or LB (with or without CSCF) and incubated for 24 h at 37° C. with shaking at 150 rpm (for B. subtilis) and non-shaking (for L. plantarum). Every 2 h, 1 mL of each sample was collected, and the optical density (OD 600) was measured using the Biowave CO8000 cell density meter.

Crystal violet biofilm quantification assay: Crystal violet biofilm quantification assays were performed in 48-well microtiter plates (Tarsons Products Pvt. Ltd. India) as previously described 41. Briefly, microbes were inoculated into respective medium and incubated overnight at 37° C. without shaking. Cultures were diluted to OD600=0.01 in fresh MRS, re-inoculated into new medium, seeded on the microtiter plates, and incubated 37° C. without shaking for 48 h and 72 h. Following incubation, the growth of the cells was measured at optical density (OD) 600 mm using a microtiter plate reader. Then, the cells attached to the surface were stained with 0.1% crystal violet for 20 min, repeatedly washed with sterile distilled water, and suspended in 95% ethanol. Plates were read at 595 nm and OD values were recorded. Graphs were represented as means±SEMs of four different trials.

Freeze-thaw challenge of L. plantarum conic colonies: An experimental flowchart for L. plantarum freeze-thaw challenge assay is depicted in FIG. 2E. Briefly, overnight culture suspensions of L. plantarum were diluted to OD600=0.01 in fresh MRS broth, spread on MRS hard agar, and incubated for 7 or 15 days at 37° C. at pH 5.5. After incubation, the plates were scaled and transferred to −17° C. for 1 h, following which the conic colonies were scraped off the plate with sterile phosphate buffer saline (PBS, pH: 7.4). The cells in PBS were vortexed vigorously for 5 min and sonicated for 2 min (10 s pulse on/off) at 4° C. with 40% amplitude to break any colony clumps. The samples were visualized under a microscope to make sure there were no clumps in the solution. Then, the cells were incubated at 37° C. and periodically scrutinized under the microscope every 15 min till 3 h for cellular aggregation. The cells were stained with Filmtracer™ LIVE/DEAD™ biofilm viability kit (Thermofishers scientific, US) and visualized under Nikon fluorescent microscope (Nikon Eclipse Ti2, Japan). The quantification of the images was performed using ImageJ V 1.8.0.

Cold-Shock Colony Filtrate (CSCF) Preparation and Assay with Unstressed Conic Colonies of L. plantarum

An assay was developed to see if the cold-stressed conic colony (−17° C. for 1 h) extracts could potentiate consolidated bundling in unstressed conic colony cells. Briefly, 1 mL of suspension from harvested cold-stressed (treated) and unstressed (control) conic colonies (approximately 5-6 colonies) were resuspended in PBS, vortexed, and sonicated. The extracts were filtered using a 0.2 μm membrane filter to remove the cells. The filtrate was named cold-shock colony filtrates (CSCF). Simultaneously, 1 mL cells from the unstressed colonies were vortexed, sonicated, and pelleted. To these pelleted unstressed cells, 1 mL of CSCF was added, aspirated, and incubated at 37° C. for 3 h. At the same time, colony filtrates (CF) from the unstressed colony and heat-treated (60° C. for 30 min) CSCF were prepared and tested. Following incubation, the aggregation pattern was scrutinized under Nikon fluorescent microscope (Nikon Eclipse Ti2, Japan). As a negative control, one vial of unstressed cells was incubated with 5M lithium chloride (LiCl2) for 30 min before adding 1 mL of CSCF. LiCl2 abolishes the S-layer protein and stalls the ability of the cells to aggregate to form bundles.

pH tolerance assay: L. plantarum tolerance to pH was studied by growing the bacteria in acidic or alkaline pH gradients ranging from pH 3.5 to 7. Briefly, MRS liquid media and hard agars were prepared, and the pH was adjusted either with 0.1 M NaOH (for alkaline pH) or IM HCl (for acidic pH). MRS liquid media with different pH settings were used for assessing the growth profiles on 50-mL Tarson tubes (Tarsons Products Pvt. Ltd. India). Biofilm formation on polystyrene plates was analyzed, while the hard agars were used to scrutinize the formation of conic colonics.

Desiccation stress experiment: Desiccation experiments were performed with 7 day old colonies grown on MRS hard agar as previously described with slight modifications²². Briefly, L. plantarum was inoculated into liquid MRS and incubated overnight at 37° C. for 24 h. Cultures were diluted to OD600=0.01 in fresh MRS, re-inoculated into a new medium, spread on MRS hard agar plates, and incubated for 7 days at 37° C. The plates were kept open in a desiccation cabinet (MRC, Holon, Israel) at 40% relative humidity and 25° C. for 24 h. Following drying, the whole colony was lifted, suspended in sterile PBS or distilled water, aspirated, sonicated to disrupt colony clumps. Then, the cells were serially diluted and plated on MRS hard agars and incubated at 37° C. for 48 h, following which the colony-forming units (CFU) were counted and recorded.

Biofilm and reporter assays with Bacillus subtilis: B. subtilis was used as a probiotic model to examine the biofilm stimulatory effect of cold-shock colony supernatant (CSCF) extracted from L. plantarum. For pellicle formation assays, 5 μl of the bacterial suspensions (5×10⁵ CFU/mL) were pipetted into 4 mL of LB in 24-well polystyrene plates, following which plates were incubated at 30° C. for 72 h. Images of the pellicles were captured using a smartphone fitted with Leica Vario-Summilux-H1.6-3.4/16-125 ASPH cameras. B. subtilis strain YC189 (ptapA-cfp expression) was used to assess the biofilm bundles in liquid LB supplemented with or without CSCF (5% v/v and 10% v/v) using CFP filter in a Nikon fluorescent microscope (Nikon Eclipse Ti2, Japan), and YC121 (ptapA-lacZ expression) strain was used for assessing the ß-galactosidase activity as previously described⁴².

C. albicans biofilm inhibition assay with live L. plantarum: Effect of Live probiotic (L. plantarum grown at pH 3.5 or 5.5) on C. albicans biofilms were tested in C. albicans biofilm inducing conditions as previously described with slight modifications⁴³. Briefly, C. albicans biofilms were grown on polystyrene plates for 8 h at 37° C. After 8 h of incubation, the supernatant was removed, replaced with PBS (controls) or live L. plantarum cells in PBS (grown previously in MRS at pH 3.5, or 5.5), and incubated again at 37° C. for 24 h. Biofilm inhibition was then assessed microscopically after staining with SYTO® 9 dye and crystal violet quantification assay.

C. elegans co-infection assays: Wild-type C. elegans were maintained on nematode growth medium (NGM) with E. coli OP50 as the feed, and synchronization was performed as described previously³⁵. Briefly, C. elegans were collected by aspiration and bleached with 2% sodium hypochlorite and 0.5 N sodium hydroxide to get the eggs. Eggs were transferred to 48-well microliter plates and were incubated for 24 h at 22° C. for hatching. The hatched juveniles were transferred to new E. coli OP50 plates and incubated for 5-7 days to obtain adult nematode. Adults were subsequently used for toxicity, and bacterial or C. albicans colonization assays. For C. albicans infection of C. elegans, the adult nematodes, previously fed on E. coli OP50, were transferred to a C. albicans lawn on NGM agar plate for 4 h. After 4 h, the nematodes were collected in M9 buffer, pipetted into a 96-well, and survival monitored for 7 days. The treatment groups were adult nematodes previously fed with the probiotic L. plantarum lawns prepared from cultures grown at pH 5.5 or pH 3.5. The live and dead nematodes were counted under bright-field, and DAPi filter and the nematode survival rates were estimated and plotted. The images of C. elegans were acquired using a Nikon fluorescent microscope (Nikon Eclipse Ti2, Japan).

Statistical analysis: All experiments were done in triplicates and results are expressed as means±standard deviations. The student's t-test was used to determine the significances of differences between treated and non-treated samples. Statistical significance was accepted for p values<0.05, and significant changes are indicated using asterisks in figures (*p<0.05, ** p<0.01, and *** p<0.001).

### Results

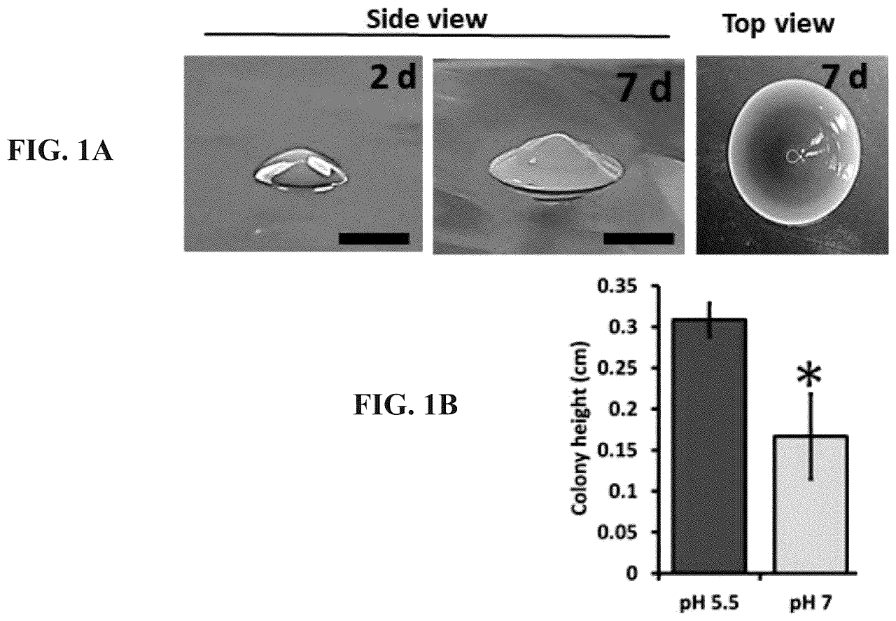

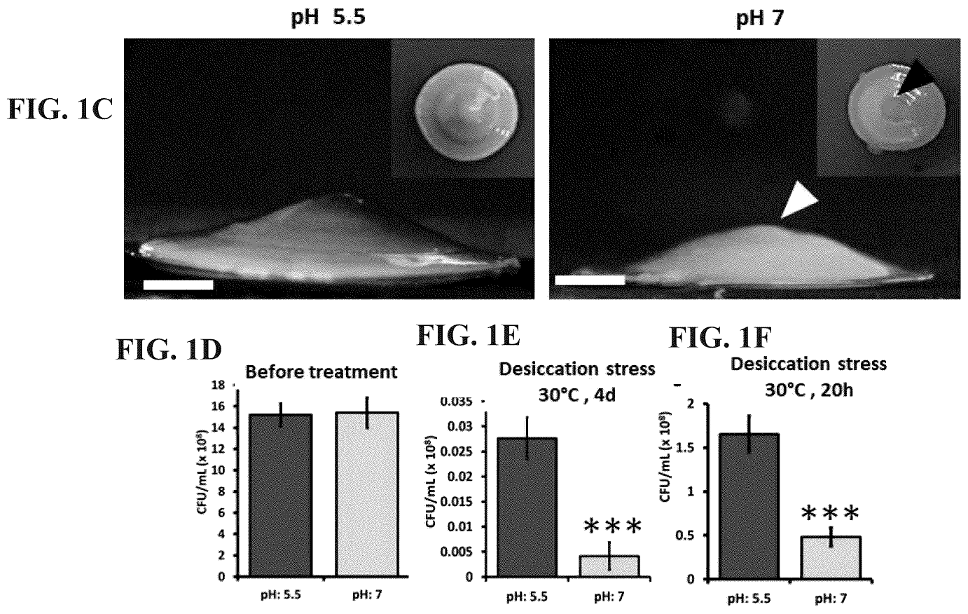

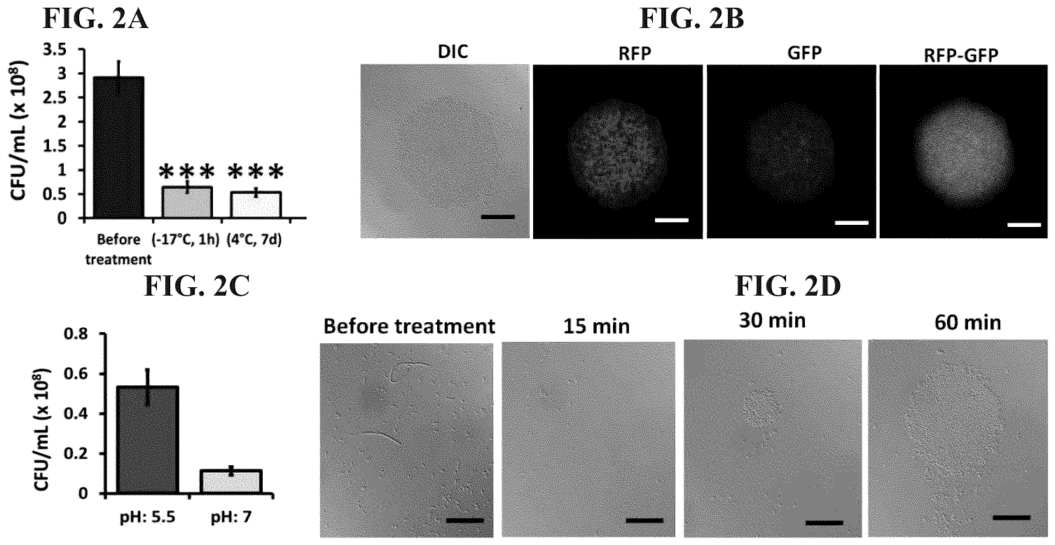

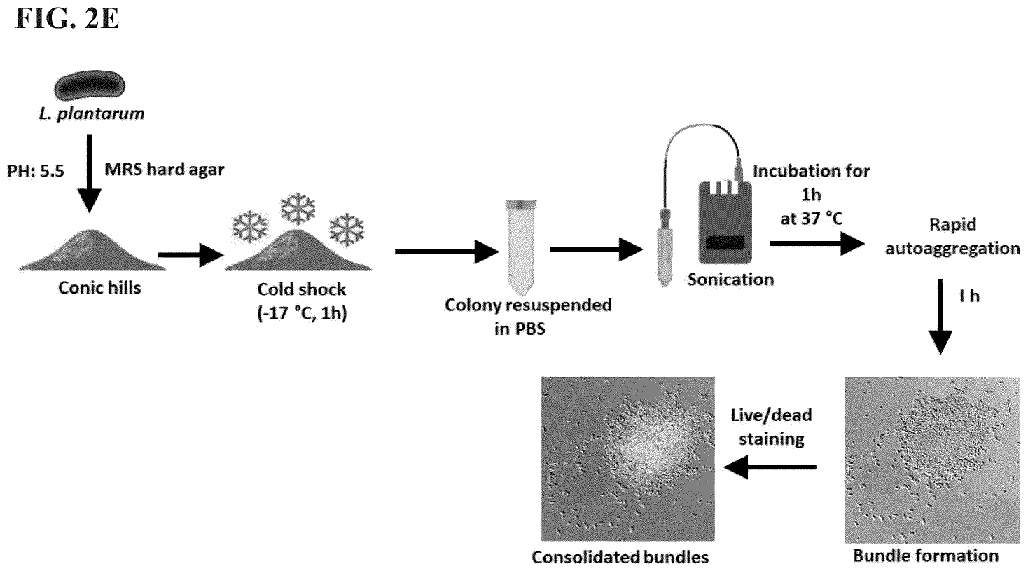

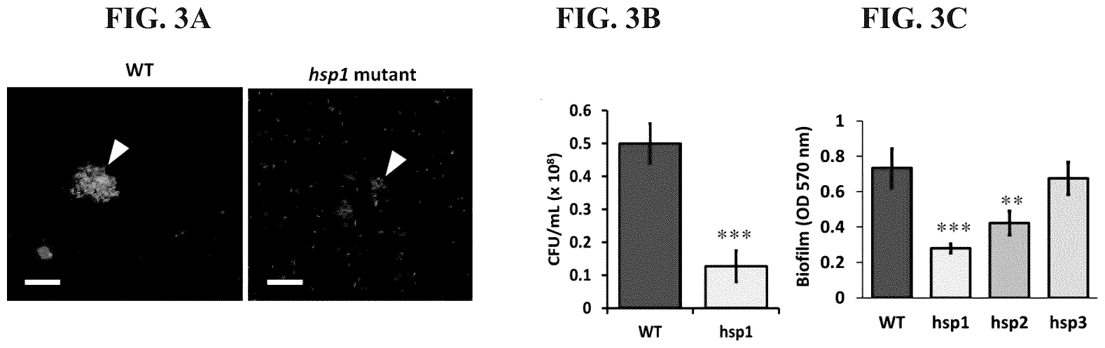

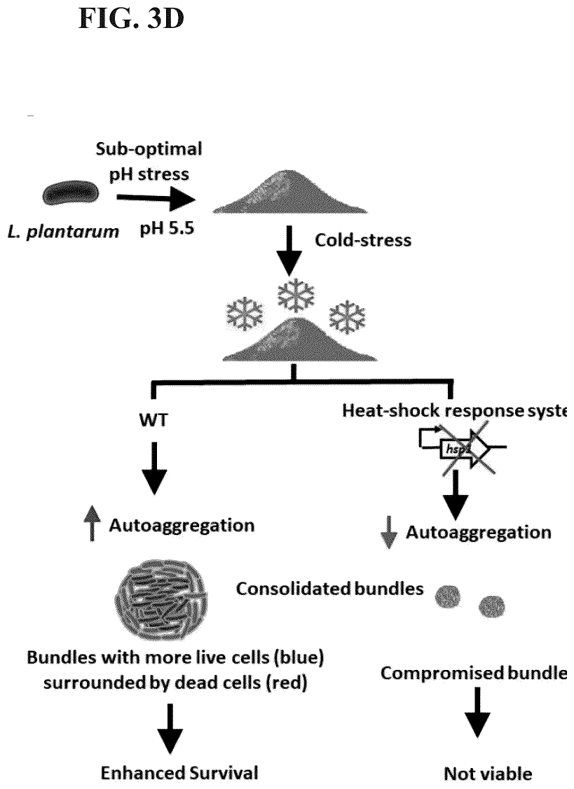

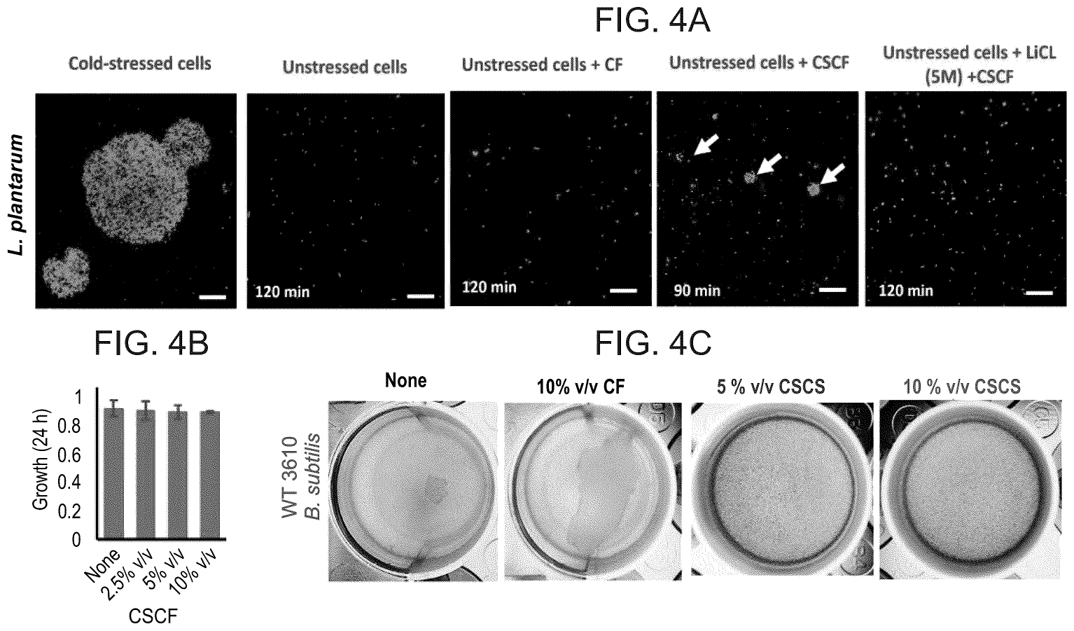

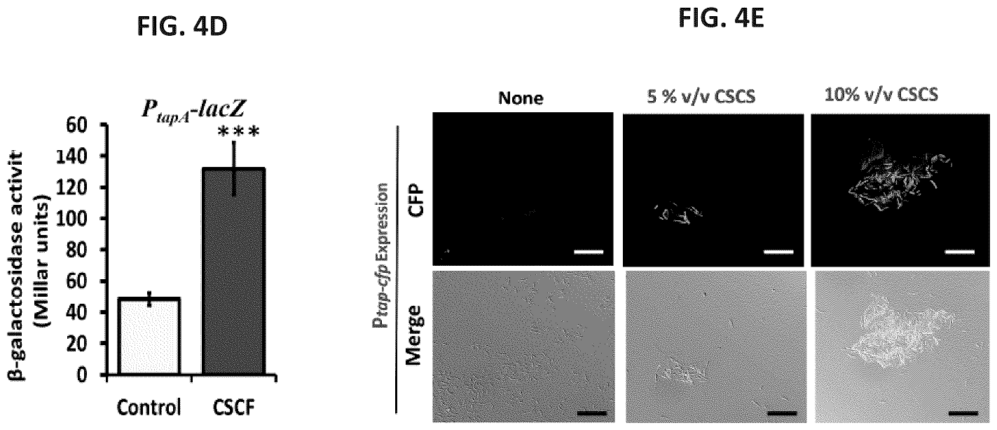

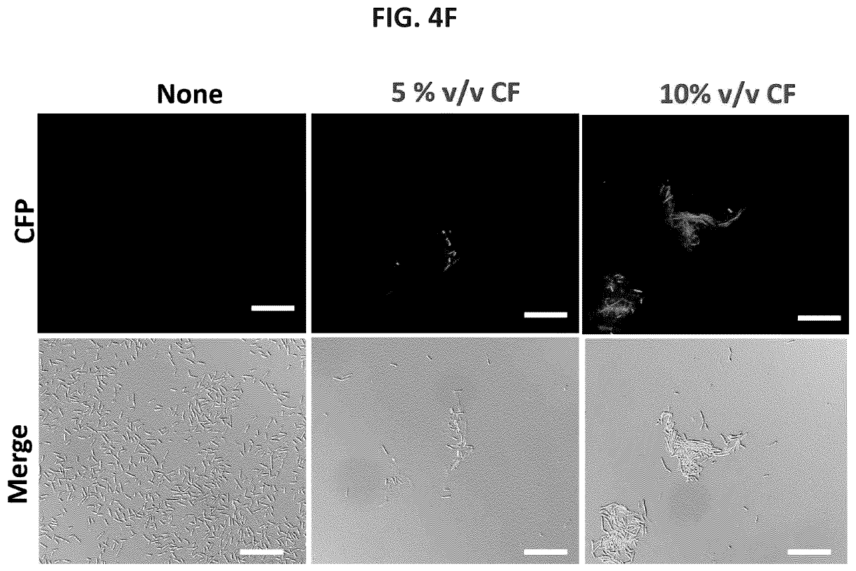

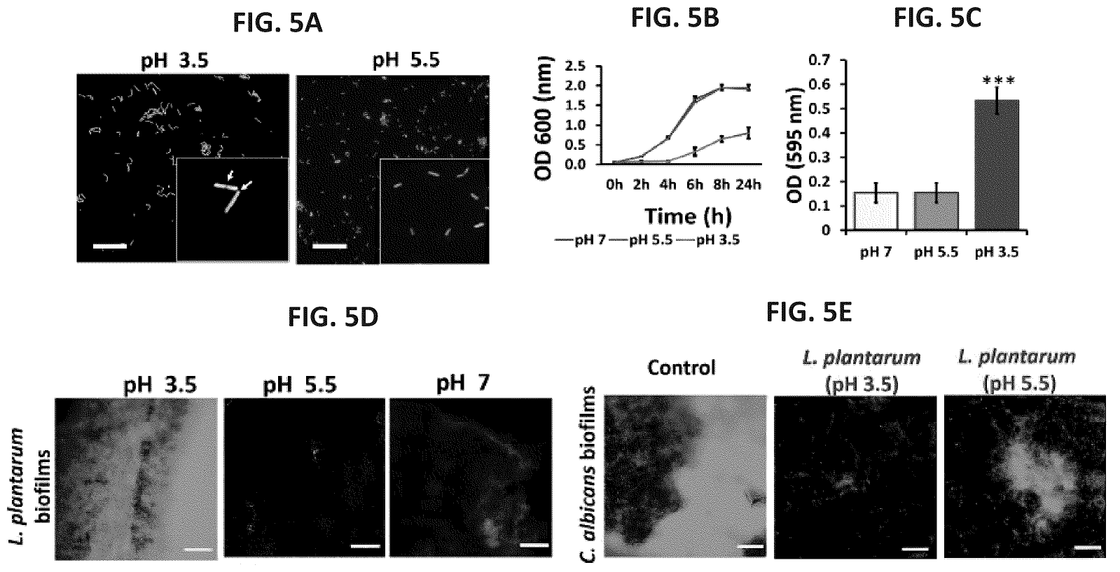

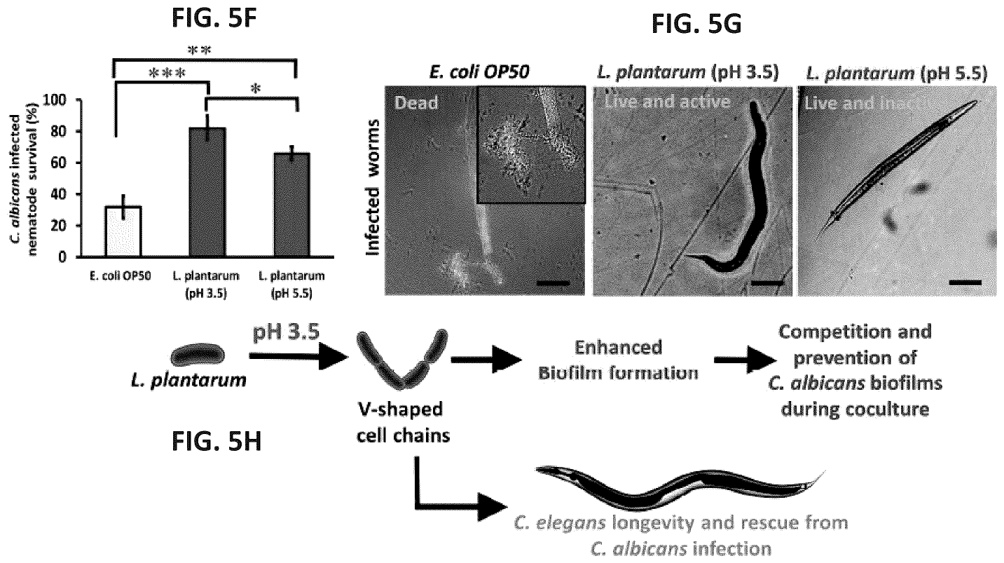

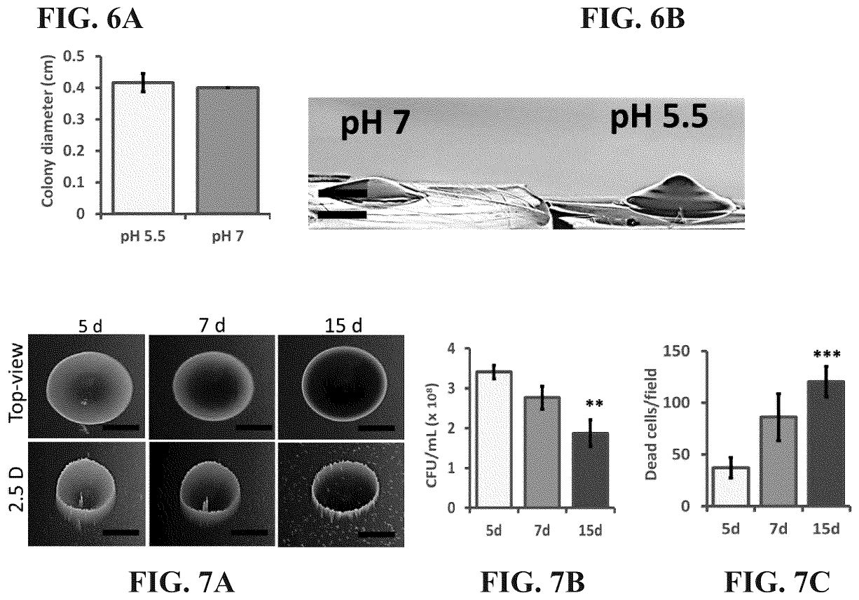

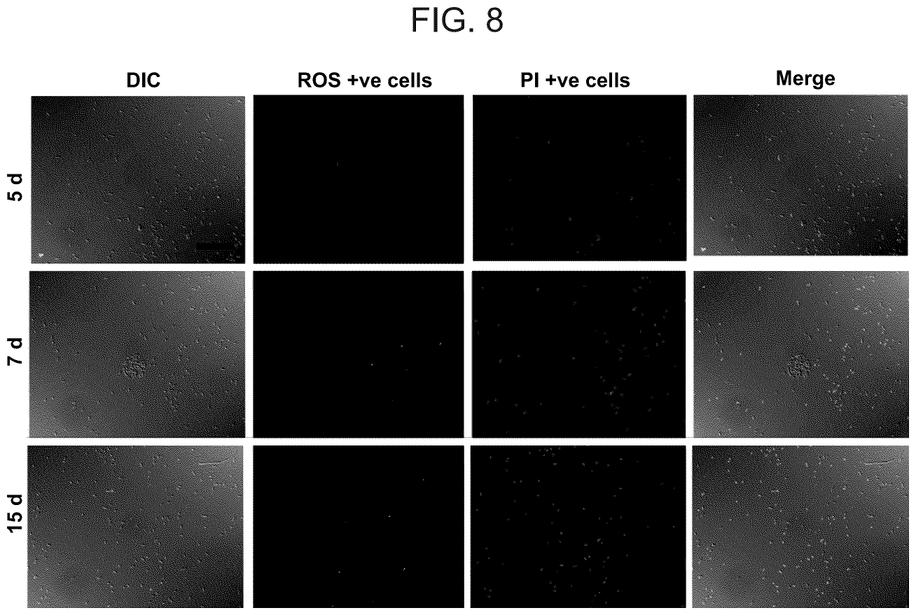

This investigation was initiated following the observation of unusual cone-shaped colonies formed by L. plantarum on the MRS air-agar interface, which was triggered by acidic pH. The highly structured small colonies expanded radially and acrially, reaching finite size with a circumferential diameter of 0.416±0.02 cm (FIGS. 6A-B); growth ceased despite the availability of nutrients in the vicinity. The most straightforward interpretation of the observed phenomenon was that the L. plantarum cells found a way for aerial expansion within the colony during the growth in acidic pH. On average, a 7d old colony measured an aerial height of 0.3 cm±0.16 cm. Brown-colored deposits were apparent on the outer surfaces of these colonies (FIG. 1A and FIG. 6B). The intensity of the brown coloration increased with incubation time (FIG. 7A), which indicated a possible correlation with cellular survivability (FIG. 7B). The numbers of dead cells were significantly higher in 15d old colonies than observed in 5 or 7d colonies (FIG. 7C). The mechanism of cell death in colonies could be attributed to ROS accumulation and instantaneous cell death (FIG. 8). Furthermore, the colonies formed at pH 7 were devoid of brown deposits and resembled a frustum without a conical tip and reduced colony height (FIGS. 1B and 1C). Despite the phenotypic heterogencities during growth in altered pH, colonies grown at acidic (pH 5.5) or alkaline (pH 7) pH had comparable viable cell counts within (FIG. 1D). These observations conclude that the brown deposits and conic tip formed at pH 5.5 are colony structures developed by the L. plantarum cells during adaptation to acidic-pH.

Having linked the cone-shaped structure and brown deposits to the acidic-pH adaptation, the present inventors next sought to understand its functional significance. They hypothesized that the observed highly structured spatiotemporal colonization would provide a multi-stress response for the bacterial population. To explore this, they exposed the colonies to secondary desiccation stress and assessed the survivability of the cells inside the colonies. The desiccation tolerance was higher in colonies grown at acidic-pH (pH 5.5) than at elevated pH (pH 7) (FIGS. 1E and 1F), signifying that the conic-shaped structure imparts some bio-shielding machinery. The possible interpretation of the observations is that brown-coloured deposits and conic shaped geometry of the colonies (grown at pH 5.5) might help the cells to improve survivability during subsequent stress.

Since cold-shock is regarded as one of the substantial stresses for the bacterial cells, the present inventors next tested whether the cone-shaped colonies would provide increased survivability during subsequent exposure to cold stress. The cellular cryotolerance drastically fell in the cold-stressed (−17° C. for 1 h) colonies (FIG. 2A), but the survivability of cells grown at the acidic-pH stressed colonies was comparatively higher (FIG. 2C). In the course of performing the cold stress experiments, several observations were made. The most intriguing finding was the sighting of unique consolidated circular bundles in acidic-pH (pH 5.5) stressed colonies following a freeze-thaw challenge (FIG. 2B). Time-lapse imaging revealed that cells harvested from the conic colony rapidly auto-aggregated to form the circular bundles (FIGS. 2D and E). Notably, the bundles consisted of live and dead cells, with the former localized at the center and the latter occupying the periphery.

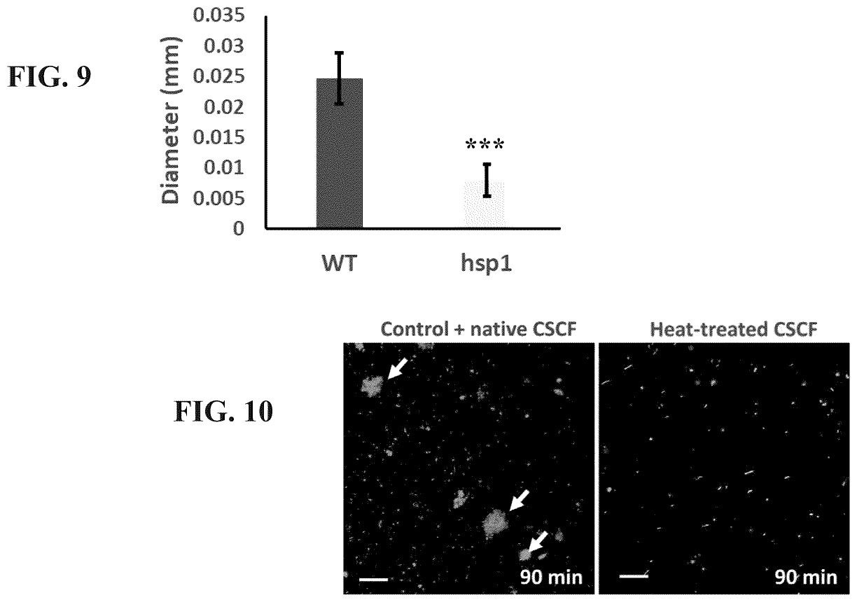

Probiotic Lactobacilli have one or two sHSP genes, with the exemption of L. plantarum that harbors three sHSP genes, namely hsp1, hsp2, and hsp3. Lately, the involvement of hsp1 in cryoprotection was stated (Arena et al., 2019). The present inventors, therefore, explored the link between sHSP and consolidated bundle formations by L. plantarum. Using hsp1 knockout mutant, they show that the bundles' size was relatively reduced (FIG. 3A and FIG. 9). Besides, hsp1 mutant also displayed reduced survivability during the freeze-thaw challenge (FIG. 3B), and formed poor biofilms on polystyrene surfaces (FIG. 3C). In contrast, hsp2 or hsp3 mutants formed either moderate or substantial biofilms, respectively, and did not show a significant difference in the freeze-thaw challenge. The findings suggest the involvement of HSP1 in governing the consolidated bundle formation and cellular protection during the cold stress (FIG. 3D).

Cold-shock colony filtrate (CSCF) was tested to see whether it could trigger circular bundle formation in unstressed colony cells. Formation of small-to-medium-sized circular bundles were noted within 90 or 120 min (FIG. 4A). The phenomenon was prevented in cells pre-incubated with lithium chloride (5M) (FIG. 4A), which removes the S-layer proteins of the bacteria, and renders the cells inefficient to receive any external signals²¹. The circular bundles were also absent when the colony filtrates (CF) from unstressed colonies (FIG. 4A) or in CSCF exposed to heat treatment (60° C. for 30 min) (FIG. 10). Overall, these results reveal that the cold stress potentiates L. plantarum cells to release pre-accumulated signals that coordinate cellular aggregation.

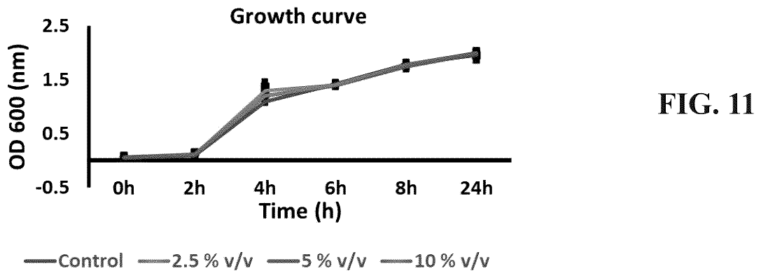

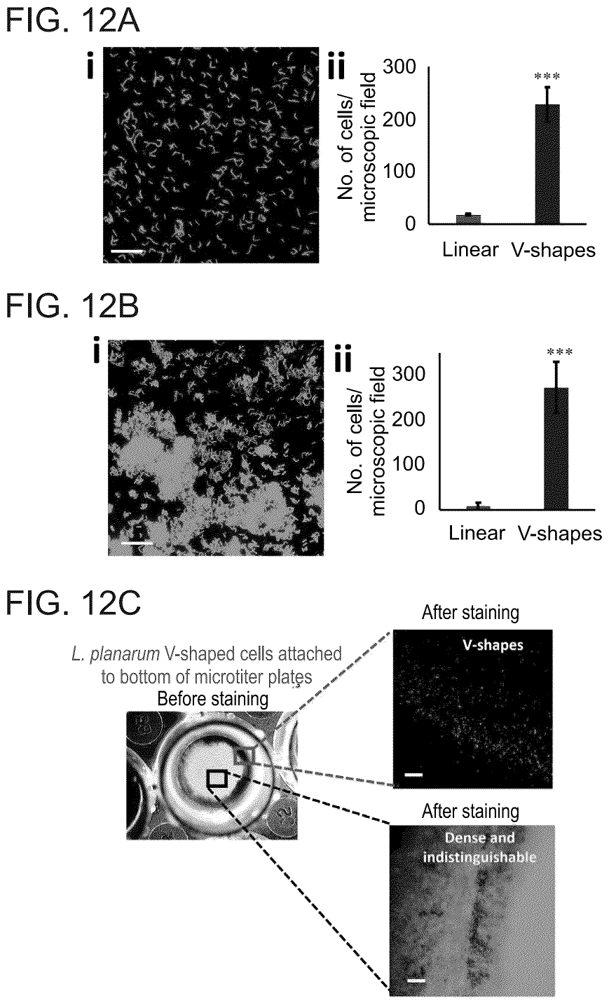

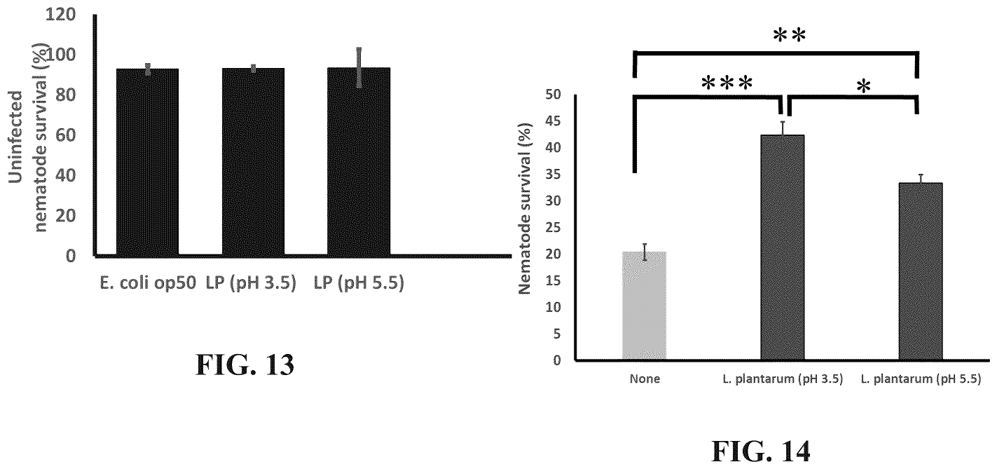

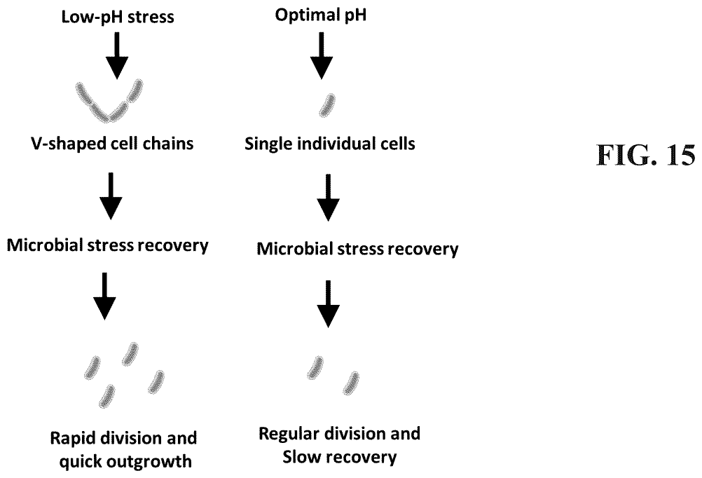

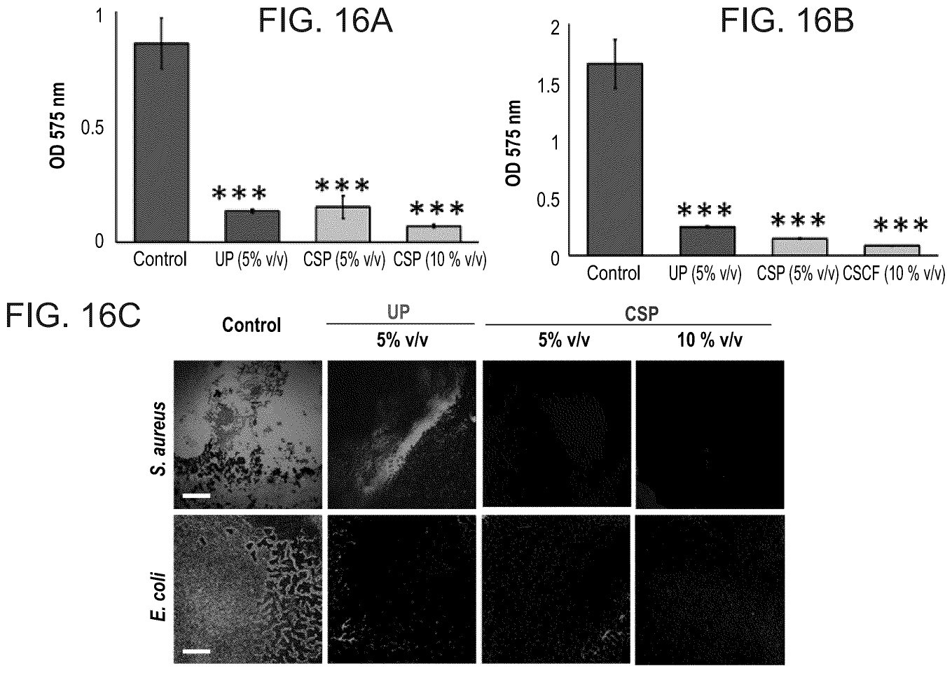

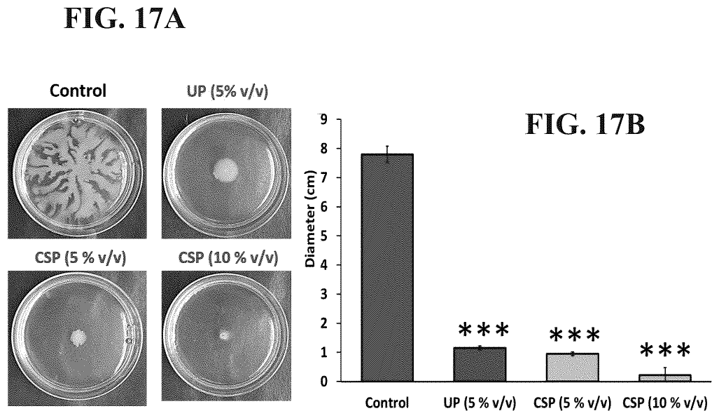

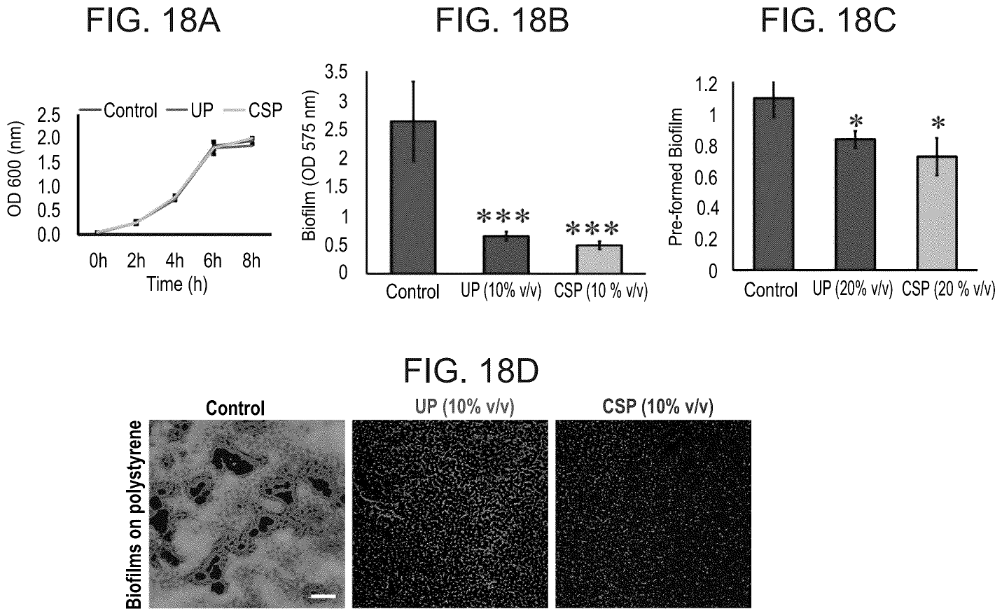

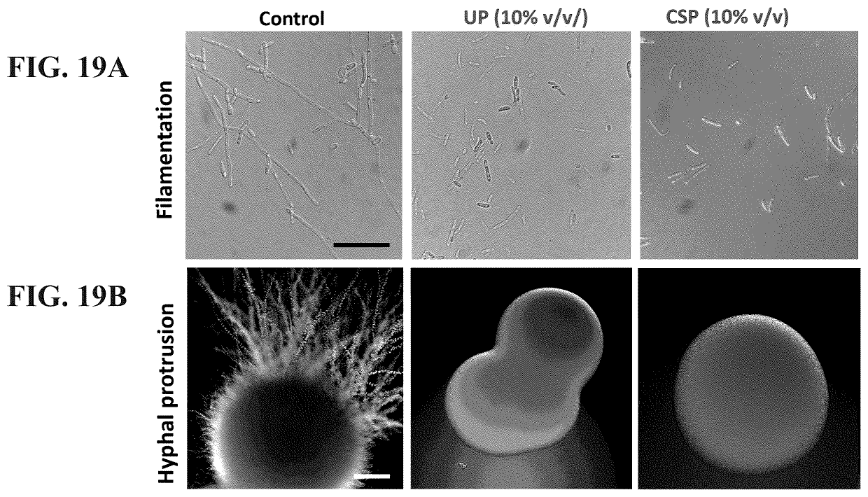

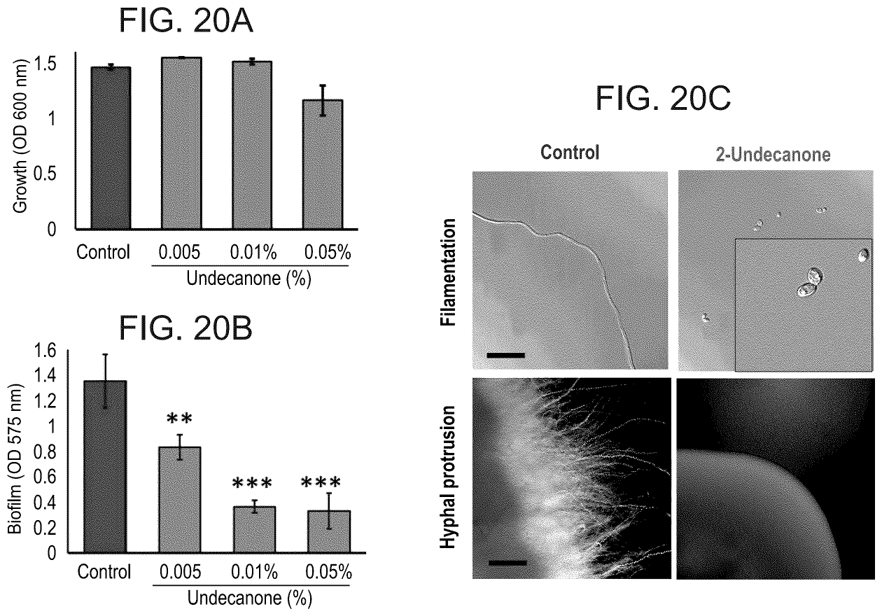

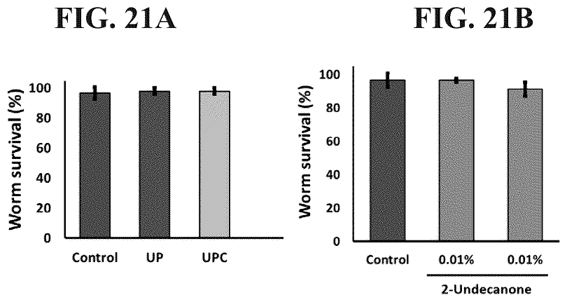

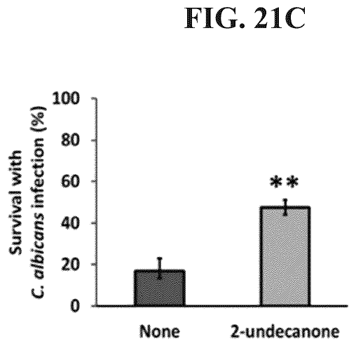

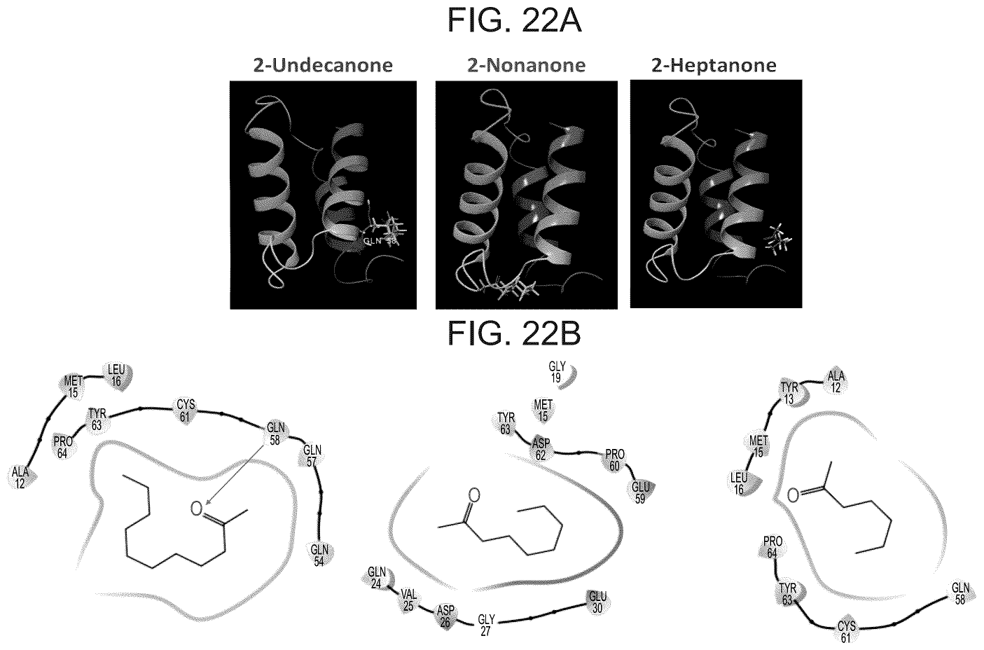

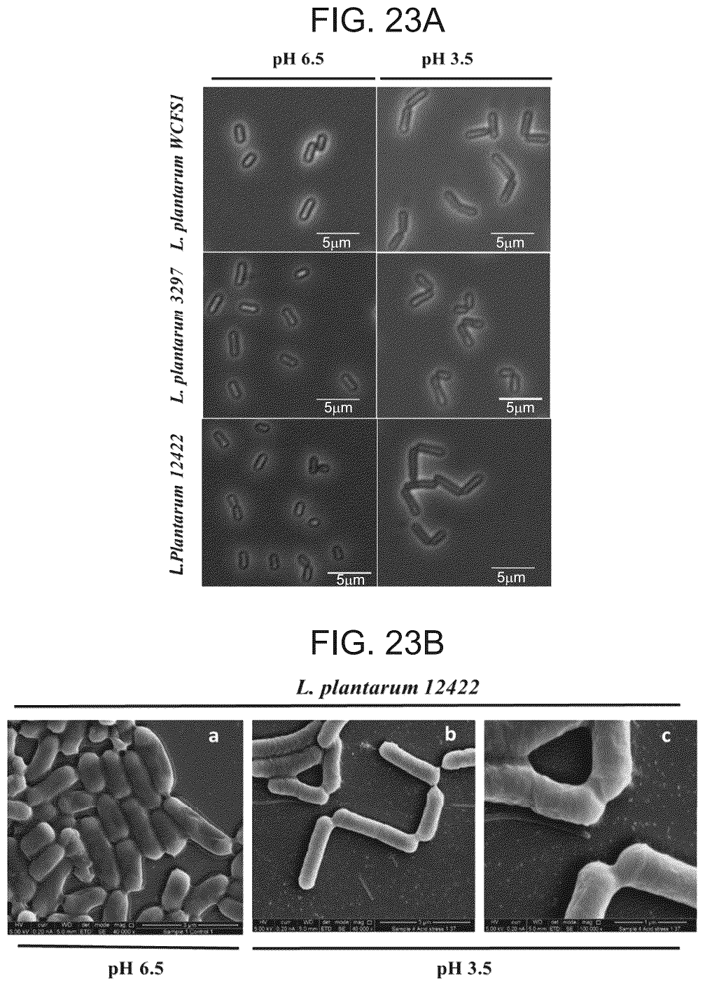

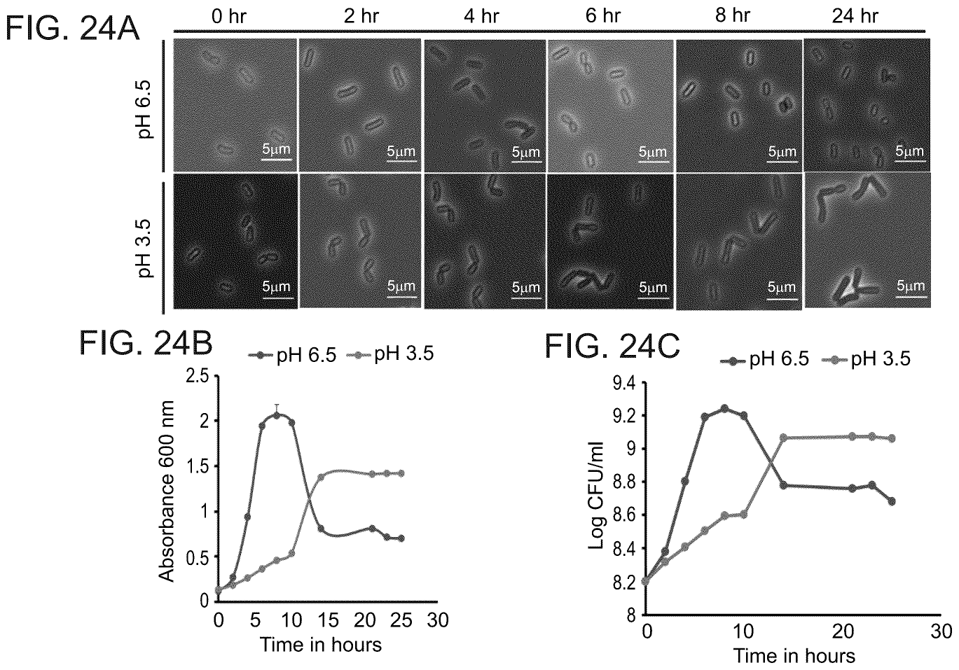

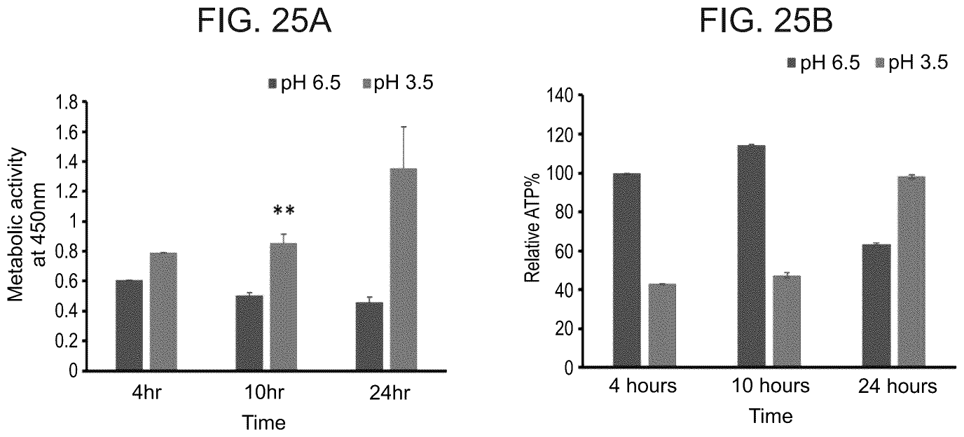

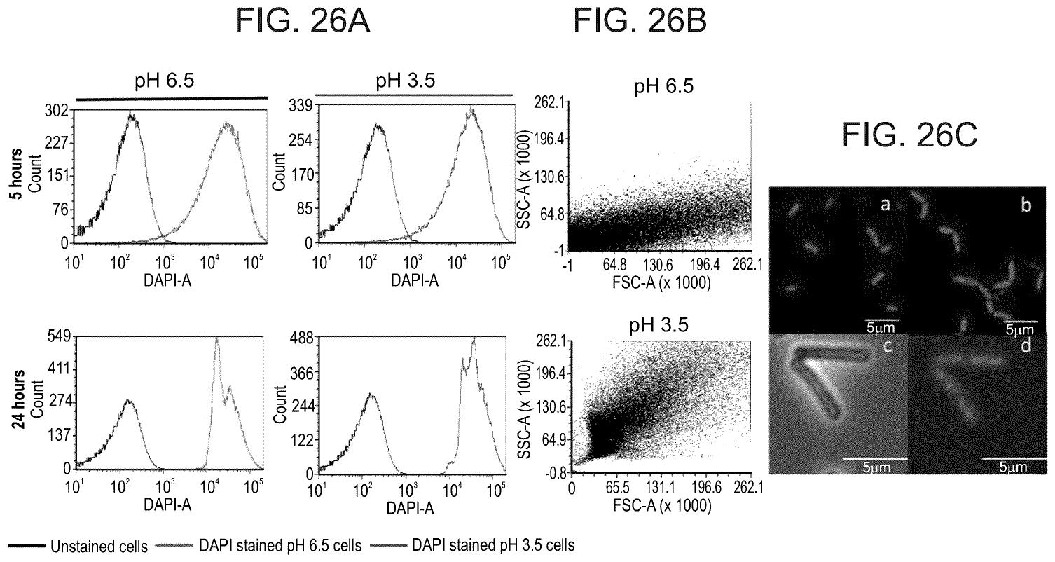

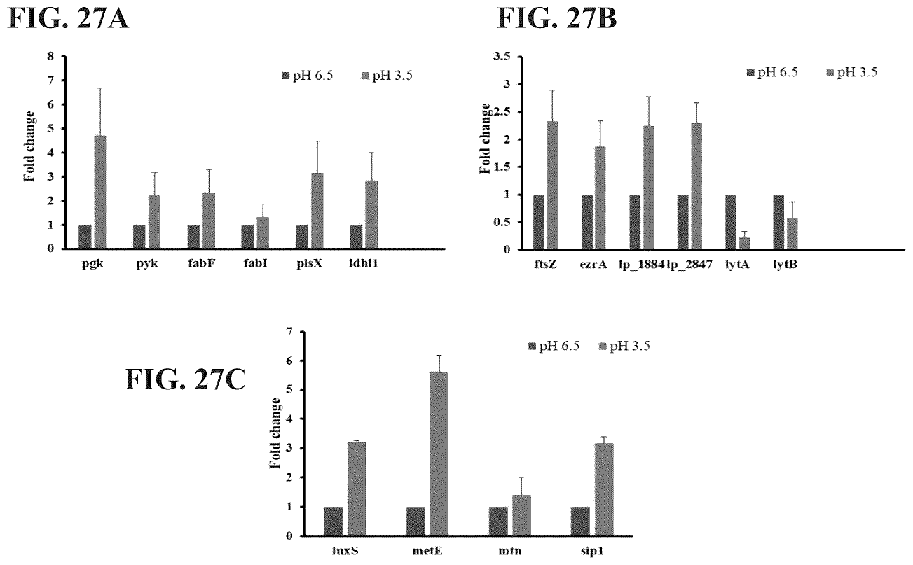

Lately, B. subtilis was shown to be involved in a symbiotic relationship with L. plantarum²². The present inventors examined supplementation of the CSCF at various doses (1-10% volume per volume (v/v)) on B. subtilis response during growth in Lysogeny broth (LB) medium. A notable induction in pellicle formation by B. subtilis at specific concentrations (5 or 10% v/v) was observed (FIG. 4C), without affecting the growth (FIG. 4B and FIG. 11). The β-galactosidase and fluorescent microscopic assays measuring the tapA (the major operon, required for polymerization of TasA amyloid fibers and their proper anchoring on cell surface towards biofilm formation) expression confirmed the activation of matrix production in response to the CSCF derived from L. plantarum cells (FIGS. 4D and 4E). Additionally, CSCF does not possess any suicidal effect on L. plantarum itself, and CF did not induce pellicle formation (FIG. 4C). The most conceivable explanation is that the putative signals are accumulated within the conic colonies and are released following cold stress disruption. The phenomenon could explain the triggering effect of the CSCF towards cellular aggregation (FIGS. 4A and E). The same result was obtained using colony filtrate of acid-stressed L. plantarum without the additional cold stress—FIG. 4F.

The adaptive morphology of L. plantarum cells during the transition to low pHs was further investigated. Intriguingly, cells grown at particularly acidic-pH (pH 3.5) displayed unique V-shaped cellular structures (FIG. 5A), though they showed slower growth rates compared to the cells grown at elevated pHs (FIG. 5B). This phenomenon was demonstrated for at least five different strains of L. plantarum. The low pH grown cells retained their V-shaped structures while forming robust biofilms on polystyrene and glass surfaces (FIGS. 5C, D and 12A-C). Collectively, these results demonstrate a survival mode of growth with increased resistance, which could be linked to biofilm formation.

It is reported that low pH (3.5-4.5) favors Lactobacilli dominance over some pathogens¹⁷. The V-shaped cells were tested to see whether they could better fight a medically important yeast pathogen, C. albicans. The effect was tested on C. albicans biofilms in in vitro and C. elegans model. L. plantarum cells (grown in media with adjusted pH 3.5, or 5.5), was added (in potato dextrose broth (PDB)) to an 8 h biofilms of C. albicans and monitored its ability to stall biofilm maturation. It was found that the live cells effectually mitigate the biofilms of C. albicans (FIG. 5E). Next, adult C. elegans were fed with L. plantarum (grown in media with either pH 3.5 or 5.5) and then infected with C. albicans. In control (E. coli OP50 fed) groups, C. albicans intensified nematode mortality rates (FIG. 5F) and effectively killed the nematode by piercing the cuticle (FIG. 5G inset). In contrast, nematodes fed with L. plantarum cells exhibited better survival rates (FIGS. 5F and G), notably the groups fed with V-shaped L. plantarum grown in medium with adjusted pH 3.5 (FIG. 5F). Uninfected nematode survival rates are shown in FIG. 13. The best interpretation for the observed phenotypes is that the L. plantarum cells likely colonize C. elegans intestine, which prevent C. albicans virulence. It also ties to the previous results that at pH 3.5 L. plantarum forms vigorous biofilms, exhibits unique V-shaped conformations (FIG. 5A, FIGS. 12A-C), and can to mitigate the colonization of C. albicans within C. elegans, and therefore enhancing C. elegans longevity.

### References for Example 1

1 Duar, R. M. et al. Lifestyles in transition: evolution and natural history of the genus Lactobacillus. FEMS Microbiol Rev. 41, S27-S48 (2017).
2 Jung, J. H. et al. Multifunctional properties of Lactobacillus plantarum strains WiKim83 and WiKim87 as a starter culture for fermented food. Food Sci Nutr. 7, 2505-2516 (2019).
3 Behera, S. S., Ray, R. C. & Zdolec, N. Lactobacillus plantarum with functional properties: an approach to increase safety and shelf-life of fermented foods. Biomed Re. Int. 2018 (2018).
4 Szlufman, C. & Shemesh, M. Role of probiotic Bacilli in developing synbiotic food: challenges and opportunities. Front Microbiol. 12, 811 (2021).
5 Giri, S. S., Sen, S. S., Saha, S., Sukumaran, V. & Park, S. C. Use of a potential probiotic, Lactobacillus plantarum L7, for the preparation of a rice-based fermented beverage. Front Microbiol. 9, 473 (2018).
6 Ramírez, M. D. F., Smid, E. J., Abee, T. & Groot, M. N. N. Characterisation of biofilms formed by Lactobacillus plantarum WCFS1 and food spoilage isolates. Int J Food Microbiol. 207, 23-29 (2015).
7 Salas-Jara, M. J., Ilabaca, A., Vega, M. & Garcia, A. Biofilm forming Lactobacillus: new challenges for the development of probiotics. Microorganisms. 4, 35 (2016).
8 Potočnjak, M. et al. Three new Lactobacillus plantarum strains in the probiotic toolbox against gut pathogen Salmonella enterica serotype Typhimurium. Food Technol Biotechnol. 55, 48-54 (2017).
9 Foysal, M. J., Fotedar, R., Siddik, M. A. & Tay, A. Lactobacillus acidophilus and L. plantarum improve health status, modulate gut microbiota and innate immune response of marron (Cherax cainii). Sci Rep. 10, 1-13 (2020).
10 Bermúdez-Humarán, L. G. et al. From probiotics to psychobiotics: live beneficial bacteria which act on the brain-gut axis. Nutrients. 11, 890 (2019).
11 Ingham, C. J., Beerthuyzen, M. & van Hylckama Vlieg, J. Population heterogeneity of Lactobacillus plantarum WCFS1 microcolonies in response to and recovery from acid stress. Appl Environ Microbiol. 74, 7750-7758 (2008).
12 Malik, S. et al. The highly autoaggregative and adhesive phenotype of the vaginal Lactobacillus plantarum strain CMPG5300 is sortase dependent. Appl Environ Microbiol. 79, 4576-4585 (2013).
13 Ferreira, C. L., GRZEŚKOWIAK, Ł., Collado, M. C. & Salminen, S. In vitro evaluation of Lactobacillus gasseri strains of infant origin on adhesion and aggregation of specific pathogens. J Food Prot. 74, 1482-1487 (2011).
14 Malik, S. et al. The highly autoaggregative and adhesive phenotype of the vaginal Lactobacillus plantarum strain CMPG5300 is sortase dependent. Appl Environ Microbiol. 79, 4576 (2013).
15 Succi, M. et al. Sub-optimal pH preadaptation improves the survival of Lactobacillus plantarum strains and the malic acid consumption in wine-like medium. Frontiers in microbiology. 8, 470 (2017).
16 Hood, S. & Zoitola, E. Effect of low pH on the ability of Lactobacillus acidophilus to survive and adhere to human intestinal cells. J Food Sci. 53, 1514-1516 (1988).
17 Zangl, I., Pap, I.-J., Aspock, C. & Schüller, C. The role of Lactobacillus species in the control of Candida via biotrophic interactions. Microbial Cell. 7, 1 (2020).
18 Martino, M. E. et al. Nomadic lifestyle of Lactobacillus plantarum revealed by comparative genomics of 54 strains isolated from different habitats. Environ Microbiol. 18, 4974-4989 (2016).
19 Guo, L. et al. Effects of lactic acid bacteria isolated from rumen fluid and feces of dairy cows on fermentation quality, microbial community, and in vitro digestibility of alfalfa silage. Front Microbiol. 10, 2998 (2020).
20 Arena, M. P. et al. The phenotypic analysis of Lactobacillus plantarum shsp mutants reveals a potential role for hsp1 in cryotolerance. Front Microbiol. 10, 838 (2019).
21 Johnson, B., Selle, K., O'Flaherty, S., Goh, Y. J. & Klaenhammer, T. Identification of extracellular surface-layer associated proteins in Lactobacillus acidophilus NCFM. Microbiology. 159, 2269 (2013).
22 Kimelman, H. & Shemesh, M. Probiotic bifunctionality of Bacillus subtilis-rescuing lactic acid bacteria from desiccation and antagonizing pathogenic Staphylococcus aureus. Microorganisms. 7, 407 (2019).
23 Warren, M. R. et al. Spatiotemporal establishment of dense bacterial colonies growing on hard agar. ELife. 8, e41093 (2019).
24 Kim, W., Racimo, F., Schluter, J., Levy, S. B. & Foster, K. R. Importance of positioning for microbial evolution. PNAS. 111, E1639-E1647 (2014).
25 Liu, S. et al. Cold-stress response of probiotic Lactobacillus plantarum K25 by iTRAQ proteomic analysis. J Microbiol Biotechnol. 30, 187-195 (2020).
26 Derzelle, S. et al. Changes in cspL, cspP, and cspCmRNA Abundance as a function of cold shock and growth phase in Lactobacillus plantarum. J Bacteriol. 182, 5105-5113 (2000).
27 Fiocco, D., Capozzi, V., Goffin, P., Hols, P. & Spano, G. Improved adaptation to heat, cold, and solvent tolerance in Lactobacillus plantarum. Appl Microbiol Biotechnol. 77, 909-915 (2007).
28 Spano, G., Capozzi, V., Vernile, A. & Massa, S. Cloning, molecular characterization and expression analysis of two small heat shock genes isolated from wine Lactobacillus plantarum. J Appl Microbiol. 97, 774-782 (2004).
29 Nguyen, P.-T., Nguyen, T.-T., Hoang, Q.-K. & Nguyen, H.-T. Response of Lactobacillus plantarum VAL6 to challenges of pH and sodium chloride stresses. Sci Rep. 11, 1-9 (2021).
30 Falagas, M. & Makris, G. Probiotic bacteria and biosurfactants for nosocomial infection control: a hypothesis. J Hosp Infect. 71, 301-306 (2009).
31 Hibbing, M. E., Fuqua, C., Parsek, M. R. & Peterson, S. B. Bacterial competition: surviving and thriving in the microbial jungle. Nat Rev Microbiol. 8, 15-25 (2010).
32 Gómez, N. C., Ramiro, J. M., Quecan, B. X. & de Melo Franco, B. D. Use of potential probiotic lactic acid bacteria (LAB) biofilms for the control of Listeria monocytogenes, Salmonella typhimurium, and Escherichia coli O157: H7 biofilms formation. Front Microbiol. 7, 863 (2016).
33 Matsubara, V. H., Wang, Y., Bandara, H., Mayer, M. P. A. & Samaranayake, L. P. Probiotic lactobacilli inhibit early stages of Candida albicans biofilm development by reducing their growth, cell adhesion, and filamentation. Appl Microbiol Biotechnol. 100, 6415-6426 (2016).
34 Köhler, G. A., Assefa, S. & Reid, G. Probiotic interference of Lactobacillus rhamnosus GR-1 and Lactobacillus reuteri RC-14 with the opportunistic fungal pathogen Candida albicans. Infect Dis Obstetr Gynecol. 2012 (2012).
35 Rajasekharan, S. K., Raorane, C. J. & Lee, J. LED based real-time survival bioassays for nematode research. Sci Rep. 8, 1-9 (2018).
36 Hunt, P. R. The C. elegans model in toxicity testing. J Appl Toxicol. 37, 50-59 (2017).
37 Scanlan, L. D. et al. Counting Caenorhabditis elegans: protocol optimization and applications for population growth and toxicity studies in liquid medium. Sci Rep. 8, 1-12 (2018).
38 Poupet, C., Chassard, C., Nivoliez, A. & Bornes, S. Caenorhabditis elegans, a host to investigate the probiotic properties of beneficial microorganisms. Front mutr. 7 (2020).
39 Breger, J. et al. Antifungal chemical compounds identified using a (′. elegans pathogenicity assay. PLOS Pathog. 3, e18 (2007).
40 Ben-Ishay, N. et al. Enrichment of milk with magnesium provides healthier and safer dairy products. NPJ Biofilms Microbiomes. 3, 1-7 (2017).
41 Rajasekharan, S. K., Lee, J.-H. & Lee, J. Aripiprazole repurposed as an inhibitor of biofilm formation and sterol biosynthesis in multidrug-resistant Candida albicans. Int J Antimicrob Agents. 54, 518-523 (2019).
42 Shemesh, M. & Chai, Y. A combination of glycerol and manganese promotes biofilm formation in Bacillus subtilis via histidine kinase KinD signaling. J Bacteriol. 195, 2747-2754 (2013).
43 James, K., MacDonald, K., Chanyi, R., Cadieux, P. & Burton, J. Inhibition of Candida albicans biofilm formation and modulation of gene expression by probiotic cells and supernatant. J Med Microbiol. 65, 328-336 (2016).

### Example 2

### Bacillary Postbiotics Suppress the Virulence of Pathogenic Yeast Through Interacting with Hyphal Wall Protein-1

### Materials and Methods

Microbial strains, and culture conditions: Microbial strains used in the study are listed in Table 1, herein above. L. plantarum was maintained and experimented in MRS hard agar (2%) or liquid medium (incubation at 37° C. without shaking), E. coli and S. aureus in Lysogeny broth (LB) (incubation at 37° C. with shaking at 150 rpm) or LB with 2% agar. C. albicans were maintained potato dextrose agar (PDA) or potato dextrose broth (PDB), and experimented in either PDA/PDB or Roswell Park Memorial Institute medium-1640 (RPMI) medium (incubation at 37° C. with shaking at 150 rpm).

Preparation of colony filtrates and pure ketones: Probiotic filtrates (postbiotics) were prepared from L. plantarum colonies grown on MRS agar plates adjusted to pH 5.5, for 5-7 days at 37° C. Briefly, L. plantarum colonies (approximately 10 similar-sized colonies) were lifted wholly and transferred to phosphate-buffered saline (PBS). The colonies were vortexed for 15 min and sonicated, following which the cells were centrifuged and pelleted at 10,000 rpm for 2 min. The supernatant was filter-sterilized using a 0.2 μm filter and designated as unstressed postbiotics (UP). For cold-stressed postbiotics (CSP) preparation, bacteria was cultured on MRS agar plates adjusted to pH 5.5 for 7 days. Then the colonies were exposed to extreme cold−17° C. for 1 h. Colonies were treated as described above for the UP preparation. Pure ketones (2-undecanone, 2-nonanonc, and 2-heptanone) were purchased from Sigma Aldrich (St. Louis, Missouri, United States).

Biofilm inhibition assay: Biofilm inhibition assays were conducted in 96-well polystyrene microtiter plates (Tarsons Products Pvt. Ltd. India) as previously described [20]. Briefly, bacterial or yeast cells were cultured overnight in LB or PDB and re-inoculated in fresh LB or PDB (1:100 dilution) (with 0.05% glucose) with and without supplementation of postbiotics (5% or 10% v/v) at 37° C. After 24 h of incubation, biofilms were stained with crystal violet (0.1%) for 20 min, washed repeatedly, and the residual biofilm cells (attached to the polystyrene surfaces) were dissolved in 95% ethanol. The planktonic growth was measured at 600 nm, while the biofilms were measured at 575 nm using a Biowave CO8000 cell density meter. For microscopic imaging of biofilms attached to the polystyrene surfaces, the wells were stained with SYTO® 9 dye, and imaged under Nikon fluorescent microscope (Nikon Eclipse Ti2, Japan) using a GFP emission filter.

Swarming motility assay: Swarming motility was assessed by spotting 2 μL of the overnight culture (E. coli) onto the center of the Petri plates containing semisolid motility agar medium (1% tryptone, 0.25% NaCl, and 0.5% agar) supplemented with or without UP or CSP (5% or 10% v/v). The plates were incubated for 37° C. for 24 h, while the subsequent branching pattern (in non-treated groups) was measured, compared (with treatment groups), and photographed using a Huawei p30 pro smartphone (Huawei VOG-L29 camera).

Yeast-hyphae switching assay: Yeast-hyphae (Y-H) switching assays were conducted in liquid RPMI-1640 medium as previously described [20]. Briefly, overnights cultures of C. albicans (grown in PDB) were diluted to a 1:100 in RPMI-1640 and treated with or without UP/CSP (5 and % v/v) or ketones (0.005%-0.1%). The cultures were then incubated at 37° C. with shaking (150 rpm). Following incubation, 5 μL of the cultures were transferred to the microscopic slide and imaged under Nikon fluorescent microscope (Nikon Eclipse Ti2, Japan).

Assay of colony morphology in hard agar: Colony morphology was generated by streaking C. albicans on PDA agar plates containing UP/CSP or ketones, and the not-treated groups. The plates were incubated at 37° C. for 7 days and the hyphal prostration from colony edges was assessed using a phase-contrast mode of the Nikon fluorescent microscope (Nikon Eclipse Ti2, Japan).

C. elegans toxicity assay: C. elegans wild-type strains were maintained on nematode growth medium (NGM) with E. coli OP50 as the feed. To assess the toxicity of UP/CSP (10% v/v) or 2-undecanone (0.01%), synchronized adult nematodes were reared and tested in a 96-well microliter plate as previously described. The control and treatment groups (each consisting of approximately 30 nematodes per well) were suspended in liquid M9 buffer and monitored for 7 days. The live or dead nematodes were counted using the Nikon fluorescent microscope (Nikon Eclipse Ti2, Japan) using a DIC and DAPi (blue LED light) filters and the survival percentage was calculated.

Molecular docking assay: Computational studies were perfumed to elucidate the interactions of methyl-3-ketones (2-undecanone, 2-nonanone, and 2-heptanone), and standard hyphal inhibitors with an Hwp1 (hyphal wall protein 1) as previously described [21] with a slight modification. The protein sequence was retrieved from UniProt (P46593) and I-TASSER online server used for the protein modelling from multiple threading alignments. The molecular docking was performed using Schrödinger Maestro 11.4 (Schrodinger Software Solutions, USA).

Statistical analysis: All experiments were done in triplicates and results are expressed as means±standard deviations. The student's t test was used to determine the significances of differences between treated and non-treated groups. Statistical significance was accepted for p values<0.05, and significant changes are indicated using asterisks in figures (*p<0.05, ** p<0.01, and *** p<0.001).

### Results

Bacillary postbiotics restrain biofilm formation by the enteropathogenic bacteria: First, the effect of differentially collected postbiotics (UP or CSP) derived from L. plantarum colonies against two bacterial pathogens (namely E. coli and S. aureus) was tested. The tested postbiotics did not show any bactericidal effect on the tested pathogens at the tested dosage. In contrast, a drastic decline in the biofilm-forming ability of the pathogen when grown in the presence of either UP or CSP (FIGS. 16A-B). Both postbiotics (UP or CSP) showed similar effects, while the CSP showed slightly better activity (marginally significant). Further, the inhibition of biofilm surface area on a polystyrene substrate by fluorescent microscopic analysis was observed (FIG. 16C). The results point that the tested postbiotics might contain putative signals (metabolites) that disarm pathogens without compromising the planktonic cells. Such molecules could prerequisite to the antibiofilm drug discovery, as they do not apply selection pressure on pathogens, thus diminishing the prospect of developmental drug-resistant variants.

The Bacillary postbiotics inhibit dendritic swarming pattern in E. coli: One of the mechanisms controlling biofilm formation is related to swarming motility regulated by the quorum-sensing (QS) system [21,22][23]. Bacteria slide on semi-solid agar to generate different types, including a dendritic pattern of swarming motility [23]. The swarming motility is a QS controlled phenotype that is usually driven by flagellated motion on semi-solid surfaces [24]. The effect of the collected postbiotics was tested on E. coli cells characterized with a dendritic pattern of swarming motility (FIGS. 17A-B). The cells, however, failed to produce the dendrite pattern when UP or CSP were added to the semi-solid agar (FIGS. 17A-B). This finding hints at the ability of the postbiotics to restrain a QS-controlled phenotypes in E. coli, revealing its possible broad-spectrum antipathogenic effects.

The Postbiotics Mitigate Candida albicans Biofilms

C. albicans is a remarkable pathogenic yeast model that displays multifarious phenotypes like biofilms, hyphae, filaments, and flocculation [20]. This species has been often used as a model for studying the antipathogenic activities of desired chemical, synthetic or natural products. The present inventors first sought to assess the biofilm formation ability of C. albicans in the presence of collected Bacillary postbiotics. As expected, UP or CSP did not possess a notable fungicidal effect on C. albicans (FIG. 18A), but showed a substantial reduction in the biofilm formation on polystyrene surfaces as quantified by crystal violet staining (FIG. 18B). The biofilm inhibitory activities of UP or CSP against the pathogen using a fluorescent staining and microscopic imaging assays was confirmed (FIG. 18D). Though the postbiotics were effectual in preventing the formation of biofilms, they were not strong enough to disassemble the pre-formed mature biofilms at the tested dosage (10% v/v). However marginal reduction in mature 24 h biofilms were noted when the concentration of postbiotics was doubled (FIG. 18C). This illustrates the dose-dependent antibiofilm activity of the tested postbiotics.

The Postbiotics Prevent Yeast-to-Hyphal Switching and Hyphal Protrusion from a Colony

The yeast-to-hyphal (Y-H) switching is dimorphic plasticity displayed by C. albicans [20]. The hyphal mode is regarded as a virulence attribute, while the yeast mode is considered commensal [25]. The present inventors assessed the dimorphic switching of C. albicans in RPMI, a hyphal inducing liquid media in which C. albicans grows as an elongated hyphal filament (FIG. 19A). In UP or CSP treated groups, the filamentation were substantially reduced (FIG. 19A). The hyphal protrusion from colony edges on hard agars was assessed. Prolonged incubation (FIG. 19B) in potato dextrose (PDA) hard agars resulted in hyphal protrusions in control groups, which were absent in treated groups (FIG. 19B). The treated groups showed smooth round colony phenotypes indicating that the tested postbiotics are efficient in suppressing filamentation in hard agars as well.

Pure Ketone Molecules Constituting the Collected Postbiotics Refrain C. albicans Physiology

L. plantarum is known to secrete diverse volatile compounds [26], which were shown to possess antibacterial and/or antifungal activities [27]. GC-MS analysis revealed the presence of volatile methyl-2-ketones derived from the L. plantarum colonies, namely: 2-undecanone, 2-nonanone, and 2-heptanopne. In an effort to identify a specific ketone molecule with antimicrobial activity, the effect of pure ketones on C. albicans physiology was tested. The following ketone molecules, 2-nonanone, and 2-heptanopne, did not show any notable effect on either biofilms or hyphal. Nonetheless, 2-undecanone, at a lower dosage (0.005%), was successful in preventing the biofilm formation of C. albicans (FIG. 20B) without compromising the planktonic growth (FIG. 20A). At higher dosage (0.05 or 0.01%), it showed a substantial decline in the growth profiles as well in RPMI media. Further, 2-undecanone effectively controlled Y-H transitions in liquid RPMI media (FIG. 20C). Overall, the biofilm formation, growth, and hyphal morphology were significantly hindered by 2-undecanone and the effect was dose-dependent. This confirms that methyl-2-ketones and the bacillary postbiotics from colonies might play a potent role in supressing C. albicans pathogenesis.

The Tested Postbiotics and the Ketone Molecules are Non-Toxic in a C. elegans Model

Finally, the toxicity of the Bacillary postbiotics and 2-undecanone on C. elegans. Toxicity testing in a biological system is a foremost criterion for pharmaceutics and drug development research. C. elegans is viewed as a universal model assessing the toxicities and is an effective alternative to rodents [27,28]. The postbiotics (10% v/v), and 2-undecanone (0.1%) did not induce any mortality to the tested nematodes (FIGS. 21A-B), thus regarding them as potentially safe for further investigation towards developing antibiofilm technology.

The present inventors further tested whether 2-undecanone could suppress C. albicans pathogenicity in a C. elegans model by infecting C. elegans with C. albicans. In this model, infected-C. elegans showed only 20% survival (80% fatality) after 7 days of incubation, suggesting that the infection with C. albicans is fatal for the nematodes. Interestingly, administration of 2-undecanone (0.01%) enhanced the survivability of infected C. elegans to 50% in treated groups (FIG. 21C), suggesting that 2-undecanone eliminates C. albicans induced killing of nematode, possibility by suppressing the biofilm or hyphal colonization within the nematode.

Molecular Interaction of Ketones with Hyphal Wall Protein 1

Next, molecular interactions of possible ligands (2-undecanone, 2-nonanone, and 2-heptanone) with the hyphal wall protein 1 (Hwp1) (a major hyphal cell wall protein required for C. albicans adhesion, biofilm formation and hyphal development) were assessed [21,29]. It is suggested that interactions of compounds with the active sites of Hwpl can lead protein inactivity. In the present model. 2-undecanone was able to interact with Hwpl by forming a hydrogen bond with Gln 58 amino acid residue in the active site of Hwpl (FIGS. 22A-B). Other ketones analysed (2-nonanone, and 2-heptanone) did not form any hydrogen bonds and revealed poor binding interactions. The results support the present in vitro observations (antibiofilm and anti-hyphal activities) with the ketones (FIG. 20C), thus largely confirming the mode of action of 2-undecanone.

### References for Example 2

1. Donlan, R. M. Biofilms: microbial life on surfaces. Emerging infectious diseases 2002, 8, 881.
2. Muhammad, M. H.; Idris, A. L.; Fan, X.; Guo, Y.; Yu, Y.; Jin, X.; Qiu, J.; Guan, X.; Huang, T. Beyond risk: bacterial biofilms and their regulating approaches. Frontiers in microbiology 2020, 11, 928.
3. Jamal, M.; Ahmad, W.; Andleeb, S.; Jalil, F.; Imran, M.; Nawaz, M. A.; Hussain, T.; Ali, M.; Rafiq, M.; Kamil, M. A. Bacterial biofilm and associated infections. Journal of the Chinese Medical Association 2018, 81, 7-11.
4. Cavalheiro, M.; Teixeira, M. C. Candida biofilms: threats, challenges, and promising strategies. Frontiers in medicine 2018, 5, 28.
5. Olsen, I. Biofilm-specific antibiotic tolerance and resistance. European Journal of Clinical Microbiology & Infectious Diseases 2015, 34, 877-886.
6. Danese, P. N. Antibiofilm approaches: prevention of catheter colonization. Chemistry & biology 2002, 9, 873-880.
7. Roy, R.; Tiwari, M.; Donelli, G.; Tiwari, V. Strategies for combating bacterial biofilms: A focus on anti-biofilm agents and their mechanisms of action. Virulence 2018, 9, 522-554.
8. Gómez, N. C.; Ramiro, J. M.; Quecan, B. X.; de Melo Franco, B. D. Use of potential probiotic lactic acid bacteria (LAB) biofilms for the control of Listeria monocytogenes, Salmonella typhimurium, and Escherichia coli O157: H7 biofilms formation. Frontiers in microbiology 2016, 7, 863.
9. Ouwehand, A. C.; Salminen, S.; Isolauri, E. Probiotics: an overview of beneficial effects. Lactic acid bacteria: genetics, metabolism and applications 2002, 279-289.
10. Vieco-Saiz, N.; Belguesmia, Y.; Raspoet, R.; Auclair, E.; Gancel, F.; Kempf, I.; Drider, D. Benefits and inputs from lactic acid bacteria and their bacteriocins as alternatives to antibiotic growth promoters during food-animal production. Frontiers in microbiology 2019, 10, 57.
11. Zangl, I.; Pap, I.-J.; Aspöck, C.; Schüller, C. The role of Lactobacillus species in the control of Candida via biotrophic interactions. Microbial Cell 2020, 7, 1.
12. Szlufman, C.; Shemesh, M. Role of Probiotic Bacilli in Developing Synbiotic Food: Challenges and Opportunities. Frontiers in microbiology 2021, 12, 638830, doi: 10.3389/fmicb.2021.638830.
13. Behera, S. S.; Ray, R. C.; Zdolec, N. Lactobacillus plantarum with functional properties: an approach to increase safety and shelf-life of fermented foods. BioMed Research International 2018, 2018.
14. Guerrieri, E.; de Niederhäusern, S.; Messi, P.; Sabia, C.; Iseppi, R.; Anacarso, I.; Bondi, M. Use of lactic acid bacteria (LAB) biofilms for the control of Listeria monocytogenes in a small-scale model. Food Control 2009, 20, 861-865.
15. Arena, M. P.; Silvain, A.; Normanno, G.; Grieco, F.; Drider, D.; Spano, G.; Fiocco, D. Use of Lactobacillus plantarum strains as a bio-control strategy against food-borne pathogenic microorganisms. Frontiers in microbiology 2016, 7, 464.
16. James, K.; MacDonald, K.; Chanyi, R.; Cadieux, P.; Burton, J. Inhibition of Candida albicans biofilm formation and modulation of gene expression by probiotic cells and supernatant. Journal of medical microbiology 2016, 65, 328-336.
17. Danilova, T.; Adzhieva, A.; Danilina, G.; Polyakov, N.; Soloviev, A.; Zhukhovitsky, V. Antimicrobial activity of supernatant of Lactobacillus plantarum against pathogenic microorganisms. Bulletin of experimental biology and medicine 2019, 167, 751-754.
18. Kim, S. W.; Kang, S. I.; Shin, D. H.; Oh, S. Y.; Lee, C. W.; Yang, Y.; Son, Y. K.; Yang, H.-S.; Lee, B.-H.; An, H.-J. Potential of Cell-Free Supernatant from Lactobacillus plantarum NIBR97, Including Novel Bacteriocins, as a Natural Alternative to Chemical Disinfectants. Pharmaceuticals 2020, 13, 266.
19. Kang, C.-H.; Kim, Y.; Han, S. H.; Kim, J.-S.; Paek, N.-S.; So, J.-S. In vitro probiotic properties of vaginal Lactobacillus fermentum MG901 and Lactobacillus plantarum MG989 against Candida albicans. European Journal of Obstetrics & Gynecology and Reproductive Biology 2018, 228, 232-237.
20. Rajasekharan, S. K.; Lee, J.-H.; Lee, J. Aripiprazole repurposed as an inhibitor of biofilm formation and sterol biosynthesis in multidrug-resistant Candida albicans. International journal of antimicrobial agents 2019, 54, 518-523.
21. Abirami, G.; Alexpandi, R.; Durgadevi, R.; Kannappan, A.; Veera Ravi, A. Inhibitory effect of morin against Candida albicans pathogenicity and virulence factor production: An in vitro and in vivo approaches. Frontiers in microbiology 2020, 11, 2320.
22. Shemesh, M.; Pasvolsky, R.; Zakin, V. External pH is a cue for the behavioral switch that determines surface motility and biofilm formation of Alicyclobacillus acidoterrestris. Journal of food protection 2014, 77, 1418-1423, doi: 10.4315/0362-028X.JFP-13-425.
23. Kearns, D. B. A field guide to bacterial swarming motility. Nature Reviews Microbiology 2010, 8, 634-644.
24. Daniels, R.; Vanderleyden, J.; Michiels, J. Quorum sensing and swarming migration in bacteria. FEMS microbiology reviews 2004, 28, 261-289.
25. Chen, H.; Zhou, X.; Ren, B.; Cheng, L. The regulation of hyphae growth in Candida albicans. Virulence 2020, 11, 337-348.
26. Fras, P.; Campos, F. M.; Hogg, T.; Couto, J. A. Production of volatile phenols by Lactobacillus plantarum in wine conditions. Biotechnology letters 2014, 36, 281-285.
27. Liu, Y.; Chen, H.; Chen, W.; Zhong, Q.; Zhang, G.; Chen, W. Beneficial effects of tomato juice fermented by Lactobacillus plantarum and Lactobacillus casei: Antioxidation, antimicrobial effect, and volatile profiles. Molecules 2018, 23, 2366.
28. Scanlan, L. D.; Lund, S. P.; Coskun, S. H.; Hanna, S. K.; Johnson, M. E.; Sims, C. M.; Brignoni, K.; Lapasset, P.; Petersen, E. J.; Elliott, J. T. Counting Caenorhabditis elegans: protocol optimization and applications for population growth and toxicity studies in liquid medium. Sci. Rep. 2018, 8, 1-12.
29. Sharkey, L. L.; McNemar, M. D.; Saporito-Irwin, S. M.; Sypherd, P. S.; Fonzi, W. A. HWP1 functions in the morphological development of Candida albicans downstream of EFG1, TUP1, and RBF1. Journal of Bacteriology 1999, 181, 5273-5279.
30. Khiralla, G. M.; Mohamed, E. A.; Farag, A. G.; Elhariry, H. Antibiofilm effect of Lactobacillus pentosus and Lactobacillus plantarum cell-free supernatants against some bacterial pathogens. Journal of Biotech Research 2015, 6, 86.
31. George-Okafor, U.; Ozoani, U.; Tasie, F.; Mba-Omeje, K. The efficacy of cell-free supernatants from Lactobacillus plantarum Cs and Lactobacillus acidophilus ATCC 314 for the preservation of home-processed tomato-paste. Scientific African 2020, 8, e00395.
32. Zamani, H.; Rahbar, S.; Garakoui, S. R.; Afsah Sahebi, A.; Jafari, H. Antibiofilm potential of Lactobacillus plantarum spp. cell free supernatant (CFS) against multidrug resistant bacterial pathogens. Pharmaceutical and Biomedical Research 2017, 3, 39-44.
33. Sharma, A.; Srivastava, S. Anti-Candida activity of spent culture filtrate of Lactobacillus plantarum strain LR/14. Journal de mycologie medicale 2014, 24, e25-e34.
34. Naglik, J. R.; König, A.; Hube, B.; Gaffen, S. L. Candida albicans-epithelial interactions and induction of mucosal innate immunity. Current opinion in microbiology 2017, 40, 104-112.
35. Pukkila-Worley, R.; Peleg, A. Y.; Tampakakis, E.; Mylonakis, E. Candida albicans hyphal formation and virulence assessed using a Caenorhabditis elegans infection model. Eukaryotic cell 2009, 8, 1750-1758.
36. Breger, J.; Fuchs, B. B.; Aperis, G.; Moy, T. I.; Ausubel, F. M.; Mylonakis, E. Antifungal chemical compounds identified using a (′. elegans pathogenicity assay. PLOS pathogens 2007, 3, e18.
37. Haque, F.; Alfatah, M.; Ganesan, K.; Bhattacharyya, M. S. Inhibitory effect of sophorolipid on Candida albicans biofilm formation and hyphal growth. Scientific Reports 2016, 6, 1-11.
38. Reddy, D. N.; Al-Rajab, A. J. Chemical composition, antibacterial and antifungal activities of Ruta graveolens L. volatile oils. Cogent Chemistry 2016, 2, 1220055.
39. Orlanda, J. F.; Nascimento, A. Chemical composition and antibacterial activity of Ruta graveolens L. (Rutaceae) volatile oils, from São Luís, Maranhão, Brazil. South African Journal of Botany 2015, 99, 103-106.
40. Lee, J. H.; Kim, Y. G.; Khadke, S. K.; Lee, J. Antibiofilm and antifungal activities of medium-chain fatty acids against Candida albicans via mimicking of the quorum-sensing molecule farnesol. Microbial biotechnology 2021, 14, 1353-1366.
41. Nguyen, Q.-B. D.; Vu, M.-A. N.; Hebert, A. A. Insect repellents: An updated review for the clinician. Journal of the American Academy of Dermatology 2018.
42. Menu, M. IR3535 2-undecanone.
43. Bohbot, J. D.; Dickens, J. C. Insect repellents: modulators of mosquito odorant receptor activity. PLOS One 2010, 5, c12138.

### Example 3

### Characterization of the V-Shape Chains in L. plantarum

The V-Shape Chaining is Conserved in Different Strains of L. plantarum

Three different strains of L. plantarum, isolated from different origins, were cultured in either standard MRS media (pH 6.5) and pH-adjusted MRS media (pH 3.5), for 24 hours at 37° C. and observed for V-shape arrangement of cells. It was intriguing to note that the V-shape arrangement of cells is identical in all three strains (FIG. 23A) following exposure to the modified MRS media. Whereas, the cells cultured in the standard growth environment of pH 6.5 remained to exist in their initial cell morphology without any rearrangement of cells. Thus, the observed phenotype of cellular arrangement could be interpreted as a preferential mode of growth adopted by L. plantarum cells when exposed to an acidic stressed environment. Further, the SEM imaging analysis (FIG. 23B) reinforced the observation made by confocal microscopy, with alterations in the cell division sites occurring during the adaptation of the bacterial cell in acidified medium.

Morphological Adaptation of L. plantarum at pH 3.5 is Established at an Early Stage of Growth

Next, the present inventors assessed the morphological changes (FIG. 24A) alongside the growth curve (FIG. 24B) of L. plantarum in MRS media at pH 6.5 and pH 3.5 for a period of 25 hours. It was noticed that the bacteria cultured at pH 3.5 had a prolonged log phase, reaching an OD₆₀₀ 1.4 after 14 hours of incubation at 37° C. when compared to that of the cells cultured at pH 6.5, which attained a stationary phase of OD₆₀₀ 1.8 after 8 hours of incubation. However, the confocal imaging (FIG. 24A) shows that the bacterial cells start to rearrange themselves forming V-shape at pH 3.5 following 2 hours of incubation and continuing until 25 hours, whereas the cells at pH 6.5 do not reposition themselves throughout their growth. Nonetheless, the cells remain to be potentially viable even after 21 hours of growth at pH 3.5 which was confirmed by the CFU analysis (FIG. 24C).

Altered Metabolic Activity and ATP Levels of the Acid Stressed L. plantarum
Having correlated V-shape phenotypic adaptation to slow growth at pH 3.5, the present inventors further sought to evaluate the metabolic activity and ATP levels in bacteria at pH 6.5 or pH 3.5 by the XTT reduction assay and BacTiter Glo-ATP assay, respectively. To explore the overall metabolic activity of the bacterial cell at pH 3.5 over a period of 24 hours, they performed a tetrazolium reduction (XTT) assay, which uses cell viability as a marker to assess the metabolic activity of the cell. The assay is based on the reduction of the XTT (yellow-colored compound) to a bright orange formazan derivative by the metabolically active cells. The results showed that the metabolic activity of the cells at pH 3.5 gradually increased over time, while that cultured at pH 6.5 slowly retarded (FIG. 25A). With the help of Bac Titre Glo-ATP assay, they further assessed the relative ATP concentration in cell culture at pH 6.5 and pH 3.5. The results showed a steady increase in ATP concentration with growth at pH 3.5 while the ATP concentration dropped after 8 hours of growth (FIG. 25B).
The Cell Cycle Analysis of Acid-Treated Cells Shows Cellular Heterogeneity with Unequal DNA Distribution

The distribution of DNA among the bacterial cells grown at pH 6.5 and pH 3.5 was determined using the flow cytometry analysis of DAPI (4′, 6′-diamidino-2-phenylindole)-stained cells. The cell cycle analysis determined the amount of DNA per bacterium and it was observed that at low pH 3.5 stress, the bacterium appeared to have multiple peaks after 5 hours and 24-hour growth when compared to that of the cells cultured at pH 6.5, having single peak (FIG. 26A). The presence of multiple peaks correlates with the scatter plot graph that shows the uneven distribution of cells at pH 3.5, whereas that of pH 6.5 has a uniform cellular distribution, explaining the cellular heterogeneity exiting in the acid stressed cells (DNA replication occurs with incomplete cell division) (FIG. 26B). This data is supported by the statistical analysis of the geometric mean of the cells stained with DAPI (Table 2), which indicated that there was a 41.28% rise in the DAPI intensity of the cells grown at pH 3.5, whereas there was a 12.89% drop in the intensity of pH 6.5 DAPI stained cells. With confocal imaging analysis of DAPI-stained cells (FIG. 26C), it was further shown that there were 8-DAPI-stained regions in a single V-shape phenotype (FIG. 26C (d)), indicating an overall accumulation of DNA in acid-adapted V-shape L. plantarum that could possibly suggest an alteration in the cell division machinery when exposed to low pH settings.

TABLE 2
Effect of pH 3.5 on the DNA content per bacterium-
Intensity of DAPI stained cells

| | | |
|---|---|---|
| | Collection | The geometric mean |
| Sample | time | of DAPI stained cells |

| | | | |
|---|---|---|---|
| L. plantarum at pH 6.5 | 5 | hours | 17139.56 |
| | 24 | hours | 1514.66 |
| L. plantarum at pH 3.5 | 5 | hours | 27200.19 |
| | 24 | hours | 38430.33 |

### Effect of Acid Stress on the Gene Expression

Expression levels of genes involved in (1) global cell metabolism (2) cell division machinery (3) quorum sensing and two-component systems were analyzed. The gene expression studies were normalized to 3 internal housekeeping, recA, dnaG and rpoD. List of genes and their functions are listed in Table 3.

### 1. Global Cell Metabolism

Transcriptional levels of genes involved in carbohydrate catabolism which aids in the production of ATP in cells grown at pH 3.5, including phosphoglycerate kinase (pgk), pyruvate kinase (pyk) and lactate dehydrogenase (ldhl1) were significantly upregulated 4.7, 2.25- and 2.83-fold, respectively when compared to the control cells (cells at pH 6.5) (FIG. 27A). These enzymes are involved in the catalytic conversion of fructose 6-phosphate and phosphoenolpyruvic acid to fructose 1,6-diphosphate and pyruvic acid, respectively. Also, ldhl1 encodes for the enzyme lactate dehydrogenase, which helps in the conversion of lactic acid to pyruvic acid. In response to acid stress, there seems to be upregulation in the genes involved in fatty acid metabolism, with significant upregulation of 3.16-fold change of plsX, which encodes for phospholipid synthesis protein and 2.33-fold change of fabF, which helps in fatty acid chain elongation (FIG. 27A). Also there is a significant upregulation of metE gene which regulates the methionine biosynthesis. These results indicate that L. plantarum activates certain metabolic pathways during the morphological adaptation process which in turn helps in the survivability of the cells.

TABLE 3

| Genes | Function |
|---|---|
| Metabolic genes | |
| 1. pgk | Glucose metabolism |
| 2. pyk | Glucose metabolism |
| 3. fabF | Fatty acid chain elongation |
| 4. fabI | Fatty acid biosynthesis |
| 5. plsX | Phospholipid biosynthesis |
| 6. ldhl | Conversion of lactate to pyruvic acid |
| 7. metE | Methionine biosynthesis |
| Cell cycle genes | |
| 1. ftsZ | Important gene in Elongasome complex, |
| | septum formation prior to cell division |
| 2. ezrA | Encodes early cell division protein |
| 3. lp_1884 | Encodes extracellular protein with lysM |
| | peptidoglycan binding domain |
| 4. lp_2847 | LysM domain with transglycosylase activity |
| 5. lytA | Cell wall hydrolase, maintaining cell shape |
| 6. lytB | Cell wall hydrolase, determines timing of |
| | division, septum maturation and lateral |
| | positioning of the septum |
| Quorum sensing genes | |
| 1. luxS | Biosynthesis of AI2 |
| 2. mtn | Biosynthesis of AI2 |
| 3. sip1 | Maturation of AI2 |
| 4. metE | Biosynthesis of AI2 |
| House-keeping genes | |
| 5. recA | |
| 6. dnaG | |
| 7. rpoD | |

### 2. Cell Cycle Genes

A strong reduction in autolysin genes was observed including lytA, which is responsible for cell shape maintenance and lytB, which regulates the timing of cell division, septum maturation and lateral positioning (lytB affects the elongasome complex), specifically ftsZ function. Studies have shown that LytB is a key player in the cell division process of L. plantarum, having two acidophilic LysM binding domain, Lp_1884 and Lp_2847 suggesting that LytB is localized close to the cell membrane when the pH of the external environment is low. A strong upregulation of lp_1884 and lp-2847 and ftsZ was also observed (FIG. 27B). This indicates that the activation of LysM domain may help in localizing lytB near the membrane at low pH, however, due to downregulation of lytB, affects the septum maturation and hence this reasoning supports incomplete cell division due to upregulation of ftsZ, which is responsible for septum formation before cell division. FIG. 26C also supports this reasoning as to incomplete cell division in L. plantarum at pH 3.5

### 3. Quorum Sensing Mechanism Genes

An upregulation in luxS, mtn and sip1 was observed (FIG. 27C) (genes which are responsible for the synthesis and maturation of AI2 respectively). This upregulation suggests that the quorum sensing mechanism could help in managing the acid-stress response.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety

What is claimed is:

1. A method of generating a biofilm comprising a nomadic bacteria, the method comprising:
(a) culturing the nomadic bacteria in an acidic environment under conditions that promote generation of a V-type structure of the nomadic bacteria; and subsequently
(b) culturing said nomadic bacteria having a V-type structure on an adherent surface, thereby generating the biofilm comprising the nomadic bacteria.

2. The method of claim 1, wherein said nomadic bacteria are of the Lactobacillus genus.

3. The method of claim 2, wherein said nomadic bacteria are of a species selected from the group consisting of L. rhamnosus, L. plantarum and L. casei.

4. The method of claim 3, wherein said nomadic bacteria is of the L. plantarum species and said acidic environment is between pH 2.5-pH 4 and/or said nomadic bacteria is of the L. casei species and said acidic environment is between pH 3.5-pH 6.5.

5. The method of claim 1, further comprising culturing an additional bacteria on said adherent surface so as to generate a biofilm comprising the nomadic bacteria and said additional bacteria.

6. The method of claim 5, wherein said additional bacteria comprises Bacillus subtilis.

7. A biofilm generated according to the method of claim 1.

8. A composition comprising isolated, nomadic bacteria, wherein at least 30% of the bacteria have a V-shaped structure.

9. The composition of claim 8, wherein said isolated, nomadic bacteria are of the Lactobacillus genus.

10. The composition of claim 9, wherein said isolated, nomadic bacteria are of a species selected from the group consisting of L. rhamnosus, L. plantarum and L. casei.

11. A method of reducing biofilm formation of a pathogen comprising:
(a) culturing a nomadic bacteria in a medium subjected to at least one stress selected from the group consisting of a pH stress, a temperature stress, an oxidative stress, an osmotic stress and a combination thereof to generate a conditioned medium; and
(b) contacting said pathogen with said conditioned medium under conditions that reduce biofilm formation of said pathogen, thereby reducing biofilm formation of the pathogen.

12. The method of claim 11, wherein said combination comprises an acidic stress and a cold stress.

13. The method of claim 11, wherein said culturing promotes a chain structure of said nomadic bacteria.

14. The method of claim 11, wherein said pathogen is a pathogenic bacteria or a fungus.

15. The method of claim 11, wherein said nomadic bacteria are of a species selected from the group consisting of L. rhamnosus, L. plantarum and L. casei.

16. A conditioned medium generated by culturing nomadic bacteria in a medium subjected to at least one stress selected from the group consisting of a pH stress, a temperature stress, an oxidative stress, an osmotic stress and a combination thereof.

17. The conditioned medium of claim 16, wherein said culturing is effected on a non-adherent surface.

18. The conditioned medium of claim 16, wherein said nomadic bacteria are of a species selected from the group consisting of L. rhamnosus, L. plantarum and L. casei.

19. The conditioned medium of claim 16, wherein said at least one stress comprises an acid stress and/or a cold stress.

20. The conditioned medium of claim 16, comprising 2-undecanone.
